(19) United States
(12) Patent Application Publication
(10) Pub. No.: US 2025/0332361 A1
(43) Pub. Date: Oct. 30, 2025

(54) PRESSURE REGULATOR DEVICE

(71) Applicant: Fisher & Paykel Healthcare Limited, Auckland (NZ)

(72) Inventor: David John Sims, Auckland (NZ)

(21) Appl. No.: 18/567,220

(22) PCT Filed: Jun. 21, 2022
(86) PCT No.: PCT/IB2022/055731

§ 371 Date: Dec. 5, 2023

Related U.S. Application Data
(60) Provisional application No. 63/202,689, filed on Jun. 21, 2021.

(51) Int. Cl. A61M 16/00

(52) U.S. Cl. CPC A61M 16/0003; A61M 2016/0027; A61M 2205/3348

(57) ABSTRACT

An apparatus for securing a pressure control tube in a user-defined position within a pressure regulator device is described. The apparatus has one or more engagement features and a releasable tube retaining means comprising a resiliently deformable locking member. The resiliently deformable locking member is configured to be releasably engageable with at least one of the engagement features such that engagement between the locking member and engagement feature causes resistance to movement of the pressure control tube within the pressure regulator device. The locking member can be at least partially disengaged from the engagement feature such that resistance to movement of the pressure control tube within the pressure regulator device is reduced.

### FIELD OF THE INVENTION

The present disclosure relates to a pressure regulator for use with a breathing assistance apparatus, such as for regulating the pressure of gases supplied to a patient from a Positive End Expiratory Pressure (PEEP) apparatus. It further relates to a pressure regulator having an apparatus for retaining a pressure control tube in a user-defined position within the pressure regulator.

### CROSS-REFERENCE TO RELATED APPLICATION

This application claims priority to U.S. Provisional Patent Application No. 63/202,689, filed Jun. 21, 2021, the disclosure of which is incorporated by reference in its entirety.

### BACKGROUND

Bubble Continuous Positive Airway Pressure (CPAP) is a known form of respiratory therapy in which a patient (typically an infant) is supplied with a flow of gas via a patient interface. The flow of gas is typically provided by a gas source in the wall of a hospital or clinic, by cylinders of compressed air and/or oxygen for example during transport, or may be provided by other breathing assistance apparatus. The patient interface is connected to two conduits, which are an inspiratory conduit and an expiratory conduit. The inspiratory conduit provides gas to the patient. The expiratory conduit provides a passage for exhaled gases from the patient. The expiratory conduit is in communication with a bubble CPAP pressure regulator, which is used to set pressure. The pressure regulator may be a chamber with a column of water into which an end portion of the expiratory conduit is submerged. The exhaled gases are discharged into the pressure regulator. Discharge of the exhaled gases into the water results in bubbling of the water i.e. a bubbling effect. The patient interface is typically configured to form a seal with the patient's mouth and/or nose. Examples of sealed patient interfaces can include a nasal mask, an oral mask, a full-face mask, nasal pillows, or a cannula with sealing nasal prongs. The pressure provided by the pressure regulator device can be controlled by varying a level of submergence or depth to which the end portion of the expiratory conduit is submerged in the column of water. The pressure regulator device can be set to provide a user-defined pressure by securing the end portion of the expiratory conduit at any one of a number of predetermined heights within a chamber or tank of the pressure regulator device.

### SUMMARY

Aspects of the disclosure are summarised below. It will be noted that aspects and embodiments of the disclosure may be combined such that features and/or embodiments of one aspect may be used with features and/or embodiments of any other aspect where compatible.

The present disclosure describes an apparatus that may mitigate the risk of stress within a releasable tube retaining means causing creep and deformation of the releasable tube retaining means during extended periods of storage or transport. Mitigating the risk of creep causing deformation to the releasable tube retaining means may also reduce the risk of unintentional movement of the pressure control tube causing unintentional changes in the pressure of gases delivered to a patient by the pressure regulator device.

According to an aspect, there is provided an apparatus for retaining a pressure control tube, having one or more engagement features, in a user-defined position within a pressure regulator device. The apparatus comprises a releasable tube retaining means. The releasable tube retaining means comprises a resiliently deformable locking member. The resiliently deformable locking member is configured to be releasably engageable with at least one of the engagement features such that engagement between the locking member and engagement feature causes resistance to movement of the pressure control tube within the pressure regulator device. The releasable tube retaining means is adapted to be selectively engageable with the pressure control tube in at least one position in which the releasable tube retaining means is in a substantially unstressed state.

At least a portion of the locking member may be received in a recessed feature of the pressure control tube when the releasable tube retaining means is in the substantially unstressed state.

The portion of the locking member that is received in the recessed feature may comprise a tube engagement portion.

The tube engagement portion of the locking member may be configured to be at least partially spaced away from a surface of the recessed feature.

The tube engagement portion may comprise a hook portion that extends upwardly towards the pressure control tube in use.

The hook portion may comprise a first segment and a second segment.

The tube engagement portion may comprise a contact surface configured to mate with a surface of the one or more engagement features of the pressure control tube.

The contact surface may be configured to be at least partially spaced away from a surface of the recessed feature when the releasable tube retaining means is in the substantially un-stressed state.

The tube engagement portion may be located adjacent a distal end of the locking member.

The recessed feature may comprise an engagement feature of the pressure control tube.

The recessed feature may comprise an engagement feature having a greater depth than one or more other engagement features of the pressure control tube.

The recessed feature may comprise an uppermost engagement feature of the one or more engagement features of the pressure control tube.

The pressure control tube may be in a fully lowered positioned when the releasable tube retaining means is in the substantially unstressed state.

The releasable tube retaining means may comprise an elongate body having a coupled end and a free end. The coupled end may be configured to be coupled to a wall of a chamber of the pressure regulator device.

The coupled end may be configured to be coupled to a top wall of the chamber.

The coupled end may comprise one or more coupling portions.

The one or more coupling portions may comprise hinge hooks or tabs that are configured to be pivotably coupled to the top wall.

The coupled end may be configured to be pivotably coupled to the wall of the chamber.

The free end may comprise an actuating member.

The actuating member may be configured to be in an un-deformed position when the releasable tube retaining means is in the substantially unstressed state.

The actuating member may comprise a push tab.

The locking member may comprise an elongate member that extends downwardly from the elongate body of the tube retaining means.

The locking member may comprise a first locking member and the releasable tube retaining means may further comprise a resiliently deformable second locking member configured to be engageable with at least one of the engagement features. Engagement between the second locking member and engagement feature may cause resistance to movement of the pressure control tube within the pressure regulator device.

The second locking member may comprise a tube engagement portion.

The tube engagement portion of the second locking member may be engageable with an engagement feature of the pressure control tube when the releasable tube retaining means is in the substantially un-stressed state.

The engagement portion of the second locking member may comprise a contact surface configured to mate with a surface of the one or more engagement features of the pressure control tube.

The contact surface of the second locking member may be configured to mate with an engagement feature of the pressure control tube when the releasable tube retaining means is in the substantially un-stressed state.

The tube engagement portion of the second locking member may be located adjacent a distal end of the second locking member.

The second locking member may comprise a hook portion located adjacent the distal end.

The hook portion of the second locking member may be configured to extend away from the pressure control tube in use.

The hook portion of the second locking member may comprise a first segment and a second segment.

The first segment of the hook portion of the second locking member may form part of the engagement portion of the second locking member.

The second segment of the hook of the second locking member may form a catch for retaining the tube retaining means in an assembled position.

The releasable tube retaining means may be configured to be selectively adaptable between a first configuration and a second configuration. In the first configuration, either the locking member or both the first locking member and second locking member are in a first position in which they are engaged with at least one engagement features of the pressure control tube such that movement of the pressure control tube within the pressure regulator device is resisted. In the second configuration, the locking member or the first locking member and second locking member are in a second position in which the locking member or the first and second locking members are at least partially disengaged from the engagement feature, such that resistance to movement of the pressure control tube within the pressure regulator device is reduced.

The releasable tube retaining means may be configured to be adapted from the first configuration to the second configuration when the actuating member is actuated.

The actuating member may be configured to be actuated when it is urged towards a chamber of the pressure regulator device.

The elongate body may comprise a hinge/fulcrum located intermediate the coupled end and the free end.

The elongate body may comprise a resiliently deformable region that extends between the coupled end and the hinge/fulcrum.

The resiliently deformable region may comprise one or more spring members.

The resiliently deformable region may comprise a pair of spring members.

The resiliently deformable portion may be configured to be in an un-deformed position when the releasable tube retaining means is in the substantially unstressed state.

The elongate body may comprise a tube receiving portion configured to receive the pressure control tube in use.

The tube receiving portion may comprises an aperture located intermediate the coupled end and the free end.

A user-defined position of the pressure control of tube within the pressure regulator device may be configured to be adjusted by applying a first predetermined downstroke force or a first predetermined upstroke force to the pressure control tube when the releasable tube retaining means is adapted to the first configuration. The user-defined position of the pressure control tube may also be configured to be adjusted by applying a second predetermined downstroke force or a second predetermined upstroke force to the pressure control tube when the releasable tube retaining means is adapted to the second configuration. The first predetermined downstroke force may be greater than the second predetermined downstroke force, and the first predetermined upstroke force may be greater than the second predetermined upstroke force.

The first downstroke force is greater than the first upstroke force and the second downstroke force is greater than the second upstroke force.

According to a further aspect, there is provided an apparatus for securing a pressure control tube in a user-defined position within a pressure regulator device. The apparatus comprises a pressure control tube comprising one or more engagement features, and a releasable tube retaining means. The releasable tube retaining means comprises a resiliently deformable locking member configured to be releasably engageable with at least one of the engagement features. The releasable tube retaining means may be selectively adaptable between: a first configuration in which the locking member is in a first position wherein the locking member is engaged with at least one of the engagement features such that movement of the pressure control tube within the pressure regulator device is resisted; and a second configuration in which the locking member is in a second position wherein the locking member is at least partially disengaged from the engagement feature, such that resistance to movement of the pressure control tube within the pressure regulator device is reduced.

When the releasable tube retaining means is in the second configuration, the resistance to movement of the pressure control tube may be reduced in comparison to the resistance to movement of the pressure control tube when the releasable tube retaining means is in the first configuration.

The locking member may comprise a tube engagement portion configured to be releasably engageable with at least one of the engagement features.

The tube engagement portion may be configured to be vertically aligned and in mated engagement with an engagement feature when the locking member is in the first position.

The tube engagement portion may be configured to be at least partially disengaged from the locking member when the locking member is in second position.

The tube engagement portion may be configured to be moved at least partially out of vertical alignment with an engagement feature when the locking member is in second position.

The tube engagement portion may be located at a distal end of the locking member.

The tube engagement portion may comprise a hook portion that extends inwardly towards the pressure control tube in use.

The tube engagement portion and the engagement features may have corresponding mating surfaces.

The locking member may comprise a first locking member. The tube retaining means may comprise a second resiliently deformable locking member configured to be releasably engageable with at least one of the engagement features.

The second locking member may be configured to be in a first position when the tube retaining means is in the first configuration; and in a second position when the tube retaining means is in the second configuration.

The second locking member may comprise a tube engagement portion.

The tube engagement portion of the second locking member may be configured to be vertically aligned and in mated engagement with an engagement feature when the second locking member is in the first position.

The tube engagement portion of the second locking member may be configured to be at least partially disengaged from the locking member when the locking member is in the second position.

The tube engagement portion of the second locking member may be configured to be moved at least partially out of vertical alignment with the engagement feature when the locking member is in the second position.

The tube engagement portion of the second locking member may be located at a distal end of the second locking member.

The second locking member may comprise a hook portion configured to extend away from the pressure control tube in use.

The hook portion of the second locking member may form at least a part of the tube engagement portion of the second locking member.

The hook portion of the second locking member may comprise a first segment and a second segment.

The first segment may form part of the tube engagement portion of the second locking member.

The second segment may form a catch for retaining the tube retaining means in an assembled position.

The releasable tube retaining means may comprise an elongate body having a coupled end and a free end, and the coupled end is configured to be coupled to a wall of a chamber of the pressure regulator device.

The coupled end may be configured to be coupled to a top wall of the chamber.

The coupled end may comprise one or more coupling portions.

The one or more coupling portions may comprise hinge hooks or tabs that are configured to be pivotably coupled to the wall or top wall of the chamber.

The coupled end may be configured to be pivotably coupled to the wall or top wall of the chamber.

The locking member or the first and second locking members may comprise elongate members that extend downwardly from the elongate body of the tube retaining means.

The free end may comprise an actuating member.

The actuating member may comprise a push tab.

The actuating member may be configured to selectively adapt the releasable tube retaining means between the first and second configurations.

The actuating member may be configured to adapt the releasable tube retaining means from the first configuration to the second configuration when an actuation force is applied to the actuating member.

The actuation force may be configured to urge the actuating member towards the wall of the chamber.

The actuating member may be fully depressed towards the wall of the chamber when the releasable tube retaining member is in the second configuration.

The elongate body may comprise a hinge/fulcrum located intermediate the coupled end and the free end.

The actuating member may be configured to be pivotable about the hinge/fulcrum.

The elongate body may comprise a resiliently deformable region that extends between the coupled end and the hinge/fulcrum.

The resiliently deformable region may comprise one or more elongate spring members.

The resiliently deformable region may comprise a pair of elongate spring members.

The resiliently deformable region may be configured to lie substantially flush with a surface of the wall or top wall of the chamber, when the releasable tube retaining means is in the first configuration.

The resiliently deformable region may be configured to be deformed and/or bow away from a surface of the wall or top wall of the chamber, when the releasable tube retaining means is in the second configuration.

The resiliently deformable region may be configured to lift or urge the locking member or first locking member out of vertical alignment and laterally away from an engagement feature, when the resiliently deformable region is deformed or bowed away from the wall or top wall of the chamber.

The actuating member may be configured to urge the second locking member downwardly and out of vertical alignment with an engagement feature, when the releasable tube retaining means is in the second configuration.

The elongate body may comprise a tube receiving portion configured to receive the pressure control tube in use.

The tube receiving portion may comprise an aperture located intermediate the coupled end and the free end.

The one or more engagement features each may comprise a notch or groove on an outer surface of a body of the pressure control tube.

The pressure control tube may comprise a plurality of engagement features that are spaced along a length of a body of the pressure control tube.

Each of the engagement features may be configured to correspond to a pressure setting of the pressure regulator device.

Each of the engagement features may comprise an upper surface and a lower surface that are angled relative to each other and an outer surface of the body of the pressure control tube.

The locking member or first and second locking members may be configured to be engaged or in contact with both the upper and lower surface of an engagement feature when the releasable tube retaining means is in the first configuration.

The locking member or first and second locking members may be configured to be at least partially disengaged from the upper surface of an engagement feature when the releasable tube retaining means is in the second configuration.

The locking member or the first locking member may be disengaged from the lower surface of the engagement feature when the releasable tube retaining means is in the second configuration.

According to a further aspect, there is provided a releasable tube retaining means for retaining a pressure control tube in a user-defined position within a pressure regulator device. The releasable tube retaining means comprising: a resiliently deformable locking member configured to be releasably engageable with an engagement feature of the pressure control tube, to retain the pressure control tube in a user-defined position. The releasable tube retaining means is configured to be adaptable between a first configuration in which the locking member is engaged with the engagement feature and resists movement of the pressure control tube, and a second configuration in which the locking member is at least partially disengaged from the engagement feature and the resistance to movement of the pressure control tube is reduced.

When the releasable tube retaining means is in the second configuration, resistance to movement of the pressure control tube may be reduced in comparison to resistance to movement of the pressure control tube when the releasable tube retaining means is in the first configuration.

The locking member may comprise a tube engagement portion configured to be releasably engageable with at least one of the engagement features.

The tube engagement portion may be configured to be vertically aligned and in mated engagement with an engagement feature when the releasable tube retaining means is in the first configuration.

The tube engagement portion may be configured to be at least partially disengaged from the engagement feature when the releasable tube retaining means is in the is in second configuration.

The tube engagement portion may be configured to be moved at least partially out of vertical alignment with an engagement feature when the releasable tube retaining means the is in second configuration.

The tube engagement portion may be located at a distal end of the locking member.

The tube engagement portion may comprise a hook portion that extends inwardly towards the pressure control tube in use.

The tube engagement portion and the engagement features may have corresponding mating surfaces.

The locking member may comprise a first locking member. The releasable tube retaining means may further comprise a second resiliently deformable locking member configured to be releasably engageable with at least one of the engagement features.

The second locking member may comprise a tube engagement portion.

The tube engagement portion of the second locking member may be configured to be vertically aligned and in mated engagement with an engagement feature when the releasable tube retaining means is in the first configuration.

The tube engagement portion of the second locking member may be configured to be at least partially disengaged from the locking member when the releasable tube retaining means is in the second configuration.

The tube engagement portion of the second locking member may be configured to be moved at least partially out of vertical alignment with an engagement feature when the releasable tube retaining means is in the second configuration.

The tube engagement portion of the second locking member may be located at a distal end of the second locking member.

The second locking member may comprise a hook portion configured to extend away from the pressure control tube in use.

The hook portion of the second locking member may form at least a part of the tube engagement portion of the second locking member.

The hook portion of the second locking member may comprise a first segment and a second segment.

The first segment may form part of the tube engagement portion of the second locking member.

The second segment may form a catch for retaining the tube retaining means in an assembled position.

The releasable tube retaining means may comprise an elongate body having a coupled end and a free end. The coupled end may be configured to be coupled to a wall of a chamber of the pressure regulator device.

The coupled end may be configured to be coupled to a top wall of the chamber.

The coupled end may be configured to be pivotably coupled to the wall or top wall of the chamber.

The coupled end may comprise one or more coupling portions.

The one or more coupling portions may comprise hinge hooks or tabs that may be configured to be pivotably coupled to the wall or top wall of the chamber.

The locking member or the first and second locking members may comprise elongate members that extend downwardly from the elongate body of the tube retaining means.

The free end may comprise an actuating member.

The actuating member may comprise a push tab.

The actuating member may be configured to selectively adapt the releasable tube retaining means between the first and second configurations.

The actuating member may be configured to adapt the releasable tube retaining means from the first configuration to the second configuration when an actuation force is applied to the actuating member.

The actuation force may be configured to urge the actuating member towards the wall of the chamber.

The actuating member may be fully depressed towards the wall of the chamber when the releasable tube retaining member is in the second configuration.

The elongate body may comprise a hinge/fulcrum located intermediate the coupled end and the free end.

The actuating member may be configured to be pivotable about the hinge/fulcrum region.

The elongate body may comprise a resiliently deformable region that extends between the coupled end and the hinge/fulcrum.

The resiliently deformable region may comprise one or more elongate spring members.

The resiliently deformable region may comprise a pair elongate of spring members.

The resiliently deformable region may be configured to lie substantially flush with a surface of the wall or top wall of the chamber when the releasable tube retaining means is in the first configuration.

The resiliently deformable region may be configured to be deformed and/or bow away from a surface of the wall or top wall of the chamber when the releasable tube retaining means is in the second configuration.

The resiliently deformable region may be configured to lift or urge the locking member or first locking member out of vertical alignment and laterally away from an engagement feature when the resiliently deformable region is deformed or bowed away from the wall or top wall of the chamber.

The actuating member may be configured to urge the second locking member downwardly and out of vertical alignment with an engagement feature when the releasable tube retaining means is in the second configuration.

The elongate body may comprise a tube receiving portion configured to receive the pressure control tube in use.

The tube receiving portion may comprise an aperture located intermediate the coupled end and the free end.

The locking member or the first and second locking members may be configured to be releasably engageable with an engagement feature of the one or more engagement features. The engagement feature may comprise a notch or groove on an outer surface of a body of the pressure control tube.

The pressure control tube may comprise a plurality of engagement features spaced along a length of a body of the pressure control tube.

The engagement features may be configured to correspond to a pressure setting of the pressure regulator device.

Each of the engagement features may comprise an upper surface and a lower surface that are angled relative to each other and an outer surface of the body of the pressure control tube.

The locking member or first and second locking members may be configured to be engaged or in contact with both the upper and lower surface of an engagement feature when the releasable tube retaining means is in the first configuration.

The locking member or first and second locking members may be configured to be at least partially disengaged from the upper surface of an engagement feature when the releasable tube retaining means is in the second configuration.

The locking member or the first locking member may be disengaged from the lower surface of the engagement feature when the releasable tube retaining means is in the second configuration.

According to a further aspect, there is provided a pressure regulator device for use with a breathing assistance apparatus. The pressure regulator device comprises: a chamber for holding a body of liquid, in use; a pressure control tube comprising one or more engagement features; and a releasable tube retaining means configured to releasably retain the pressure control tube in a user-defined position within the pressure regulating device. The releasable tube retaining means comprises a resiliently deformable locking member configured to be releasably engageable with an engagement feature of the one or more engagement features. The releasable tube retaining means is configured to be adaptable between a first configuration and a second configuration. In the first configuration, the locking member is engaged with an engagement feature of the one or more engagement features and resists movement of the pressure control tube. In the second configuration the locking member is at least partially disengaged from the engagement feature such that the resistance to movement of the pressure control tube is reduced.

When the releasable tube retaining means is in the second configuration, resistance to movement of the pressure control tube may be reduced in comparison to resistance to movement of the pressure control tube when the releasable tube retaining means is in the first configuration.

The locking member may comprise a tube engagement portion configured to be releasably engageable with at least one of the engagement features.

The tube engagement portion may be configured to be vertically aligned and in mated engagement with an engagement feature when the releasable tube retaining means is in the first configuration.

The tube engagement portion may be configured to be at least partially disengaged from the engagement feature when the releasable tube retaining means is in the is in the second configuration.

The tube engagement portion may be configured to be moved at least partially out of vertical alignment with an engagement feature when the releasable tube retaining means the is in second configuration.

The tube engagement portion may be located at a distal end of the locking member.

The tube engagement portion may comprise a hook portion that extends inwardly towards the pressure control tube in use.

The tube engagement portion and the engagement features may have corresponding mating surfaces.

The locking member may comprise a first locking member. The releasable tube retaining means may comprise a second resiliently deformable locking member configured to be releasably engageable with at least one of the engagement features.

The second locking member may comprise a tube engagement portion.

The tube engagement portion of the second locking member may be configured to be vertically aligned and in mated engagement with an engagement feature when the releasable tube retaining means is in the first configuration.

The tube engagement portion of the second locking member may be configured to be at least partially disengaged from the locking member when the releasable tube retaining means is in second configuration.

The tube engagement portion of the second locking member may be configured to be moved at least partially out of vertical alignment with an engagement feature when the releasable tube retaining means is in the second configuration.

The tube engagement portion of the second locking member may be located at a distal end of the second locking member.

The second locking member may comprise a hook portion configured to extend away from the pressure control tube in use.

The hook portion of the second locking member may form at least a part of the tube engagement portion of the second locking member.

The hook portion of the second locking member may comprise a first segment and a second segment.

The first segment may form part of the tube engagement portion of the second locking member.

The second segment may form a catch for retaining the tube retaining means in an assembled position.

The pressure regulator device may further comprise an elongate body having a coupled end and a free end. The coupled end may be configured to be coupled to a wall of the chamber.

The coupled end may be configured to be coupled to a top wall of the chamber.

The coupled end may be configured to be pivotably coupled to the wall or top wall of the chamber.

The coupled end may comprise one or more coupling portions.

The one or more coupling portions may comprise hinge hooks or tabs that are configured to be pivotably coupled to the wall or top wall of the chamber.

The locking member or the first and second locking members may comprise elongate members that extend downwardly from the elongate body of the tube retaining means.

The free end may comprise an actuating member.

The actuating member may comprise a push tab.

The actuating member may be configured to selectively adapt the releasable tube retaining means between the first and second configurations.

The actuating member may be configured to adapt the releasable tube retaining means from the first configuration to the second configuration when an actuation force is applied to the actuating member.

The actuation force may be configured to urge the actuating member towards the wall of the chamber.

The actuating member may be fully depressed towards the wall of the chamber when the releasable tube retaining member is in the second configuration.

The elongate body may comprise a hinge/fulcrum located intermediate the coupled end and the free end.

The actuating member may be configured to be pivotable about the hinge/fulcrum.

The elongate body may comprise a resiliently deformable region that extends between the coupled end and the hinge/fulcrum.

The resiliently deformable region may comprise one or more elongate spring members.

The resiliently deformable region may comprise a pair elongate of spring members.

The resiliently deformable region may be configured to lie substantially flush with a surface of the wall or top wall of the chamber when the releasable tube retaining means is in the first configuration.

The resiliently deformable region may be configured to be deformed and/or bow away from a surface of the wall or top wall of the chamber when the releasable tube retaining means is in the second configuration.

The resiliently deformable region may be configured to lift or urge the locking member or first locking member out of vertical alignment and laterally away from an engagement feature when the resiliently deformable region is deformed or bowed away from the wall or top wall of the chamber.

The actuating member may be configured to urge the second locking member downwardly and out of vertical alignment with an engagement feature when the releasable tube retaining means is in the second configuration.

The elongate body may comprise a tube receiving portion configured to receive the pressure control tube in use.

The tube receiving portion may comprise an aperture located intermediate the coupled end and the free end.

The one or more engagement features may each comprise a notch or groove on an outer surface of a body of the pressure control tube.

The pressure control tube may comprise a plurality of engagement features spaced along a length of a body of the pressure control tube.

Each of the engagement features may be configured to correspond to a pressure setting of the pressure regulator device.

Each of the engagement features may comprise an upper surface and a lower surface that are angled relative to each other and an outer surface of the body of the pressure control tube.

The locking member or first and second locking members may be configured to be engaged or in contact with both the upper and lower surface of an engagement feature when the releasable tube retaining means is in the first configuration.

The locking member or first and second locking members may be configured to be at least partially disengaged from the upper surface of an engagement feature when the releasable tube retaining means is in the second configuration.

The first locking member is disengaged from the lower surface of the engagement feature when the releasable tube retaining means is in the second configuration.

The releasable tube retaining means may be adapted to be selectively engageable with the pressure control tube in at least one position in which the releasable tube retaining means is in a substantially unstressed state.

At least a portion of the locking member or the first locking member may be configured to be received in a recessed feature of the pressure control tube when the releasable tube retaining means is in the substantially unstressed state.

The portion of the locking member or the first locking member that is received in the recessed feature may be the tube engagement portion.

The tube engagement portion of the locking member or the first locking member may be configured to be at least partially spaced away from a surface of the recessed feature.

The recessed feature may comprise an engagement feature of the one or more engagement features of the pressure control tube.

The recessed feature may comprise an engagement feature having a greater depth than one or more other engagement features.

The recessed feature may comprise an uppermost engagement feature of the one or more engagement features.

The pressure control tube may be in a fully lowered positioned when the releasable tube retaining means is in the substantially unstressed state.

The releasable tube retaining means may be in the first configuration when in the substantially unstressed state.

A user-defined position of the pressure control of tube within the pressure regulator device may be configured to be adjusted by applying a first predetermined downstroke force or a first predetermined upstroke force to the pressure control tube when the releasable tube retaining means is adapted to the first configuration, and/or by applying a second predetermined downstroke force or a second predetermined upstroke force to the pressure control tube when the releasable tube retaining means is adapted to the second configuration. The first predetermined downstroke force may be greater than the second predetermined downstroke force. The first predetermined upstroke force may be greater than the second predetermined upstroke force.

The first downstroke force may be greater than the first upstroke force. The second downstroke force may be greater than the second upstroke force.

According to a further aspect, there is provided a pressure regulating device, comprising: a chamber configured to hold a column of liquid, a releasable tube retaining means, and a pressure control tube configured to be selectively moveable within the column of liquid to increase or decrease a pressure of the pressure regulating device.

The pressure control tube may comprise one or more engagement features spaced along a length of the pressure control tube. The releasable tube retaining means may comprise a first resiliently deformable locking member and a second resiliently deformable locking member. Each of the first locking member and the second locking member may comprise a tube engagement portion configured to be releasably engageable with at least one engagement feature.

Engagement of the tube engagement portion of the first locking member with an engagement feature of the one or more engagement features, and/or the tube engagement portion of the second locking member with an engagement feature of the one or more engagement features provides resistance to movement of the pressure control tube within the pressure regulating device and retains the pressure control tube at a user-defined depth within the column of liquid.

The releasable tube retaining means is selectively adaptable between a first configuration and a second configuration. In the first configuration, the tube engagement portions of the first locking member and the second locking member are each engaged with at least one of the one or more engagement features such that movement of the pressure control tube within the pressure regulating device is resisted by a first upstroke resistance force and a first downstroke resistance. In the second configuration, at least one of the tube engagement portions of the first locking member and the second locking member is at least partially disengaged from the engagement features, such that resistance to movement of the pressure control tube within the pressure regulator device is resisted by a second upstroke resistance force and a second downstroke resistance force.

One or both of the second upstroke and downstroke resistance force are less than the first upstroke resistance force and the first upstroke resistance force.

The releasable tube retaining means is adapted to be selectively engageable with the pressure control tube in a position in which the releasable tube retaining means is in a substantially unstressed state.

Further aspects of the disclosure, which should be considered in all its novel aspects, will become apparent from the following description.

### BRIEF DESCRIPTION OF DRAWINGS

One or more embodiments of the present disclosure will now be described by way of example(s) with reference to the accompanying drawings in which:

FIG. 1 is a block diagram showing a typical configuration for supplying breathing assistance to a patient;

FIG. 2 is a perspective view of a first embodiment of a pressure regulator device according to the present invention;

FIG. 3 is a perspective view of a top of a device or apparatus for securing a pressure control tube at a user-defined position within the pressure regulator device of FIG. 2;

FIG. 4 is a perspective view of a bottom of the device or apparatus of FIG. 3;

FIG. 5 is a perspective view of a top of a top wall or lid of the pressure regulator device of FIG. 2;

FIG. 6 is a perspective view of a bottom of the top wall of FIG. 4

FIG. 7 is a plan view of the top of the top wall of FIG. 5;

FIG. 8 is a plan view of the bottom of the top wall of FIG. 5;

FIG. 9 is a close-up partial cross-section view of the top wall of FIG. 7;

FIG. 10 is a perspective view of a pressure control tube of the device of FIG. 3;

FIG. 11 is a front view of the pressure control tube of FIG. 10;

FIG. 12 is a plan view of a top of the pressure control tube of FIG. 10;

FIG. 13 is a close-up cross-sectional view of the pressure control tube of FIG. 10

FIG. 14 is a perspective view of a releasable tube retaining means of the device of FIG. 3;

FIG. 15 is a plan view of a top of the releasable tube retaining means of FIG. 13;

FIG. 16 is a front view of the releasable tube retaining means of FIG. 13;

FIG. 17 is a plan view of the bottom of the releasable tube retaining means of FIG. 13;

FIG. 18 is a perspective view of the device of FIG. 2 with the lid cut away, with releasable tube retaining means in a first configuration;

FIG. 19 is a front view of the device of FIG. 2 with the lid cut away, with releasable tube retaining means in a first configuration;

FIG. 20 is a cross-sectional front view of the device of FIG. 2 with the lid partially cut away, with releasable tube retaining means in a first configuration;

FIG. 21 is a close-up partial view of FIG. 20;

FIG. 22 is a perspective view of the device of FIG. 2 with the lid partially cut away, with releasable tube retaining means in a second configuration;

FIG. 23 is a front view of the device of FIG. 2 with the lid partially cut away, with releasable tube retaining means in a second configuration;

FIG. 24 is a cross-sectional front view of the device of FIG. 2 with the top wall partially cut away, with releasable tube retaining means in a second configuration;

FIG. 25 is a close-up partial view of FIG. 24;

FIG. 26 is a cross-sectional front view of the device of FIG. 2 with top wall partially cut away, with pressure control tube in a fully lowered position;

FIG. 27 is a close-up partial cross-sectional view the device of FIG. 26 without the top wall, with the pressure control tube in the fully lowered position;

FIG. 28 is a perspective view of the apparatus of FIG. 2 without pressure control tube, with releasable tube retaining means in a pivoted position for assembly with the top wall;

FIG. 29 is a perspective view of the apparatus of FIG. 28, wherein the releasable tube retaining means is in another pivoted position for assembly with the top wall;

FIG. 30 is a cross-section front view of the apparatus of FIG. 28.

FIG. 31 is a top down perspective view of a device or apparatus for securing a pressure control tube at a user-defined position within the pressure regulator device of FIG. 2, including a second embodiment of a releasable tube retaining means;

FIG. 32 is a close-up cross-sectional front view of the of the device of FIG. 31;

FIG. 33. Is a perspective view of the releasable tube retaining means of FIG. 31;

FIG. 34 is a perspective view of the device of FIG. 31 without the pressure control tube, with releasable tube retaining means in a pivoted position for assembly with the top wall; and

FIG. 35 is a perspective view of the device of FIG. 31, wherein the releasable tube retaining means is in another pivoted position for assembly with the top wall.

### DESCRIPTION OF EMBODIMENTS

Throughout the description like reference numerals will be used to refer to like features in different embodiments.

A Bubble CPAP pressure regulator device can provide means of producing variations or oscillations in the pressure of gases supplied to a patient connected to a positive pressure ventilation device. By submerging the end of an exhalatory or expiratory conduit into a water column, the resulting bubbles may generate a variation or ripple in the mean pressure of gases delivered to the patient. Varying the level of depth to which the end of the exhalatory conduit is submerged within the water may also provide a simple method of varying the mean pressure of gases supplied to the patient. To keep the mean pressure of gases supplied to the patient constant, the level of submergence of the end of the exhalatory conduit may be kept constant.

The present disclosure provides an apparatus or device or means for retaining an end of an exhalatory conduit or a pressure control tube in a user-defined or user-selected position within a bubble CPAP pressure regulator device, such that a mean pressure of gases delivered to a patient is maintained at a constant level. The end of the exhalatory conduit or pressure control tube can be retained at a constant user-defined or user-selected level of submergence or depth within a column of water in a tank or chamber of a bubble CPAP pressure regulator device.

Unintentional changes in the position of the pressure control tube within a bubble CPAP pressure regulator device can cause unintentional changes in the pressure of gases delivered to patients. This can be dangerous and may damage the patient's lungs, particularly if the pressure increases. Unintended changes in the position of a pressure control tube may occur, for example, if a retention means for retaining the pressure control tube within a pressure regulator device is disengaged from the tube and the pressure control tube drops a distance into the chamber.

Pressure regulator devices can be in storage or transport for extended periods of time, during which they may be exposed to varying environmental conditions. The apparatus for securing the pressure control tube within the pressure regulator device, or components thereof, may be under stress or exposed to adverse environmental conditions during storage or transport. Such adverse environmental conditions may, for example, include fluctuating or high temperatures. Components of the apparatus may experience deformation due to creep or viscoelastic stress relaxation as a result of extended exposure to adverse conditions. Deformation of one or more of the components may result in reduced engagement between the components of said apparatus. In some configurations this may result in reduced engagement between a tube retention means and a pressure control tube. This in turn may result in the pressure control tube not being effectively retained within the chamber of the pressure regulator device during use. If the pressure control tube is not effectively retained within the pressure regulator device, it is possible that the pressure control tube could unintentionally fall or drop into the chamber and body or column of liquid, resulting in an unintended increase in pressure of gases delivered to a patient.

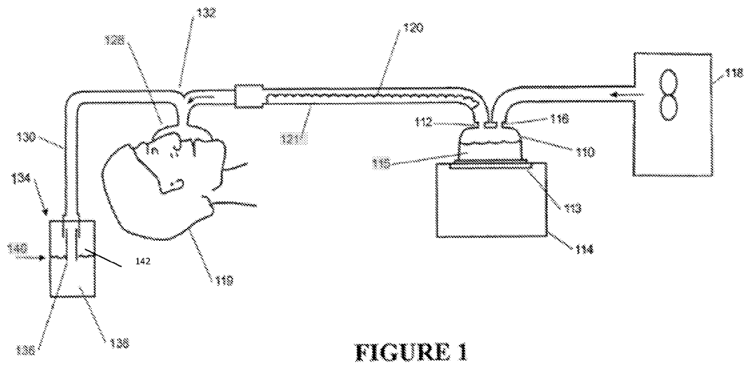

FIG. 1 depicts an example of a bubble continuous positive airway pressure (CPAP) ventilation system. A Positive End Expiratory Pressure (PEEP) system is shown in which a patient 119 is receiving gas through a patient interface, such as respiratory mask 128 or cannula connected to an inhalatory (or inspiratory) conduit 121. In the embodiment shown, the PEEP system is a humidified system and the patient is receiving humidified and pressurised gas. The present disclosure, however, is not limited to the delivery of PEEP gases but is also applicable to other types of gases delivery systems and may not necessarily involve humidification.

Humidifier 114 may comprise a heater plate 113 and a humidification chamber 110. Inhalatory conduit 121 may be connected to the outlet 112 of a humidification chamber 110 which contains a volume of water 115. Inhalatory conduit 121 may contain heating means or heater wires 120 which heat the walls of the conduit, and/or the gases within the conduit. Heating may assist to ensure a constant humidity profile along the conduit and reduce condensation of humidified gases within the conduit. As the volume of water 115 within humidification chamber 110 is heated, water vapour begins to fill the volume of the chamber 110 above the water's surface. Water vapour may be passed out of the humidification chamber 110 outlet 112 with a flow of gases (for example air) provided from a gases supply means, flow generator or blower 118 which enters the chamber 110 through inlet 116. The humidified gases may pass through the inhalatory conduit 121 to the patient interface, such as mask 128 or cannula, which typically forms a seal with the patient's 119 mouth and/or nose. Excess gases may flow through an exhalatory or expiratory conduit 130 to a pressure regulator device 134.

The pressure regulator device 134 may discharge the flow of exhalatory gases into a chamber 142. The chamber 142 may contain a column of fluid such as water 138. Gases flowing through the expiratory conduit 130 may be discharged into the body of water 138 via pressure control tube or probe 136. Pressure control tube or probe 136 may extend from the expiratory conduit 130 into the chamber 142. This results in a bubbling effect, whereby the gases eventually exit the chamber 142 via an outlet port. The outlet port can also be used to initially fill the chamber 142 with water. The outlet port may include shielding to prevent or reduce liquid aerosols created by the vigorous bubbling on the surface of the water from being expelled. It will be appreciated that the pressure control tube or probe 136, could equally be integrated into the end of the expiratory conduit 130.

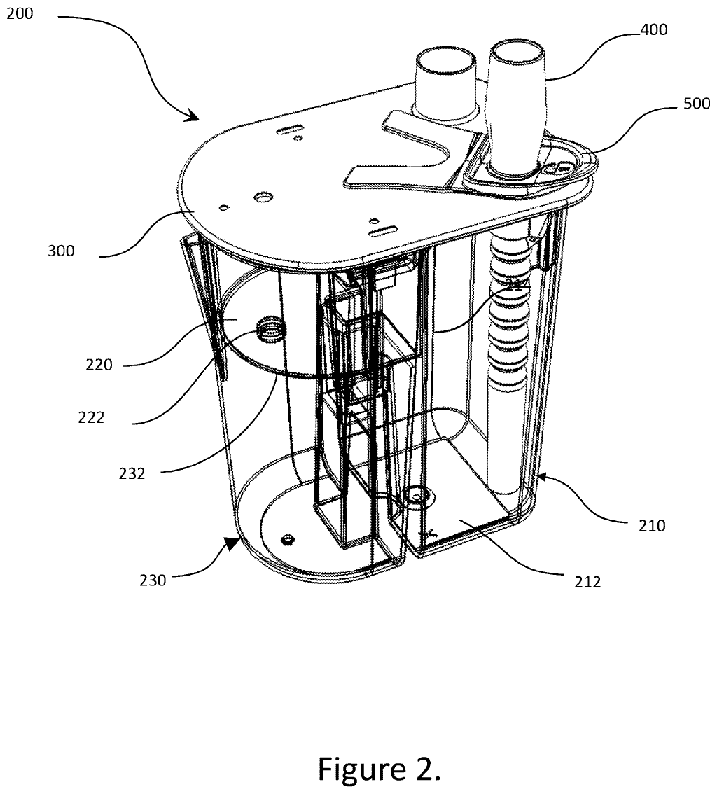

FIG. 2 depicts an example embodiment of pressure regulator device 200. In the embodiment shown, the pressure regulator device 200 is of a type which may be referred to as a bubble CPAP pressure regulator device. The pressure regulator device 200 may comprise a tank or chamber 210 with one or more side walls 214, a bottom wall 212, and a top wall 300. A probe or pressure control tube 400 may be locatable in the chamber 210. A releasable tube retaining means 500 can be provided to retain the pressure control tube 400 in a user-defined or user-selected position within the chamber 210. The pressure regulator device 200 may also include a levelling chamber 220 and an overflow facility 230.

The chamber 210 may comprise a tank, vessel or other receptacle. The chamber 210 may be configured to hold a body or column of water or other liquid, in use. The chamber 210 may comprise a bottom wall 212, one or more side walls 214 and a top wall 300. In the illustrated embodiment the chamber 210 is substantially a rectangular cuboid shape having four side walls 214. The side walls 214 may form an angle with the bottom wall 212 that is greater than 90°. A top of the chamber 210 may have a greater cross-sectional area than the bottom wall 212. The chamber 210 may comprise a filling indicator configured to indicate a maximum level to which the chamber 210 may be filled with liquid.

The levelling chamber 220 and overflow facility 230 may be configured to work in conjunction with each other to maintain the column of water or other liquid at a substantially constant level within the chamber 210. The levelling chamber 220 and the overflow facility 230 may be configured to mitigate risk of variations in the level of the water or liquid in the chamber 210 significantly altering a mean pressure of the gases delivered by the pressure regulator device 200. Variations in the water or liquid level in the chamber 210 may be caused by bubbles generated by the exhalatory gases that are delivered into the column of liquid. Variations in the water or liquid level in the chamber 210 may also be caused by humid exhalatory gases condensing when they encounter cool surfaces in the pressure regulator device 200.

The levelling chamber 220 may comprise a chamber or tank that may be fluidly coupled to the chamber 210. The levelling chamber may be configured to receive water or liquid that bubbles over from the chamber 210. The levelling chamber 220 may be upwardly open or may be at least partially enclosed. In the embodiment as shown in FIG. 2, the levelling chamber is upwardly open and comprises a top opening (not shown). The top opening of the levelling chamber 220 may be planar with a top of the chamber 210. The levelling chamber 220 may comprise an outlet or aperture 222 through which water or liquid may exit or drain from within the levelling chamber 220. The outlet 222 may be located on a bottom wall of the levelling chamber 220.

The overflow facility 230 may comprise a chamber or tank that may be fluidly coupled to the levelling chamber 220. The overflow facility 230 may be configured to receive water or liquid that has overflowed into the levelling chamber 220 from the chamber 210. The overflow facility 230 may be upwardly open and comprise a top opening 232. The overflow facility 230 may be positioned below the levelling chamber 220. The top opening 232 of the overflow facility 230 may be adjacent a bottom wall of the levelling chamber 220. Water or liquid may drain or exit from the levelling chamber 220 via the outlet or aperture 222 and flow into the overflow facility 230 via the top opening 232.

The pressure regulator device 200 can comprise an apparatus or device 600 for retaining the pressure control tube 400 in a user-defined position within the pressure regulator device 200. The apparatus 600 may be configured to retain the pressure control tube 400 in one or more discrete user-defined or user-selected positions. Each of the one or more discrete user-defined positions can correspond to a depth of submersion of the pressure control tube 400 within the body or column or liquid. The depth of submersion of the pressure control tube 400 within the body of liquid determines a pressure of gases delivered to a patient by the pressure regulator device 200. Each of the one or more discrete user-defined positions can also correspond to a pressure setting of the pressure regulator device 200.

The apparatus 600 can comprise a wall of the chamber 210 and a releasable tube retaining means 500. The releasable tube retaining means 500 may be configured to engage with or couple to the wall of the chamber 210. The releasable tube retaining means 500 may be configured to engage with the pressure control tube 400 such that the pressure control tube 400 can be releasably retained relative to the wall of the chamber 210. The tube retaining means 500 may be coupled to a side wall 214 or top wall 300 of the chamber 210.

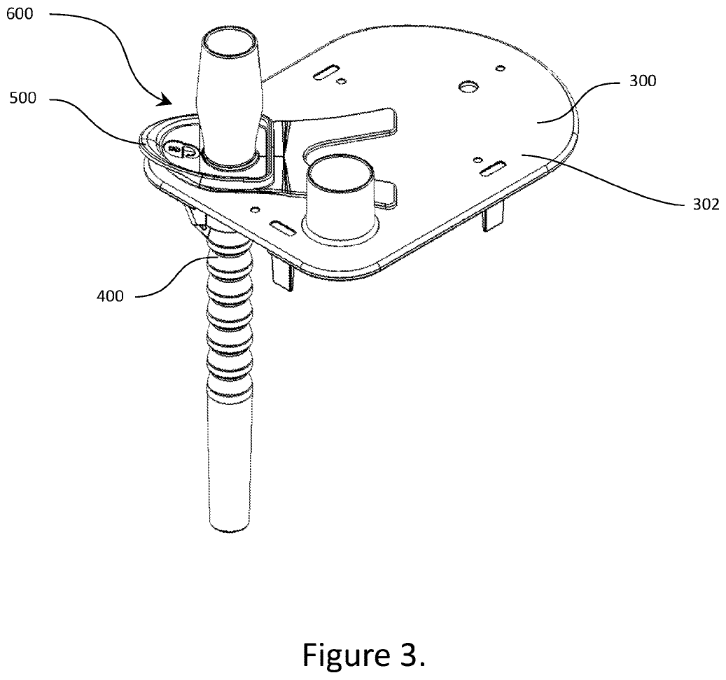

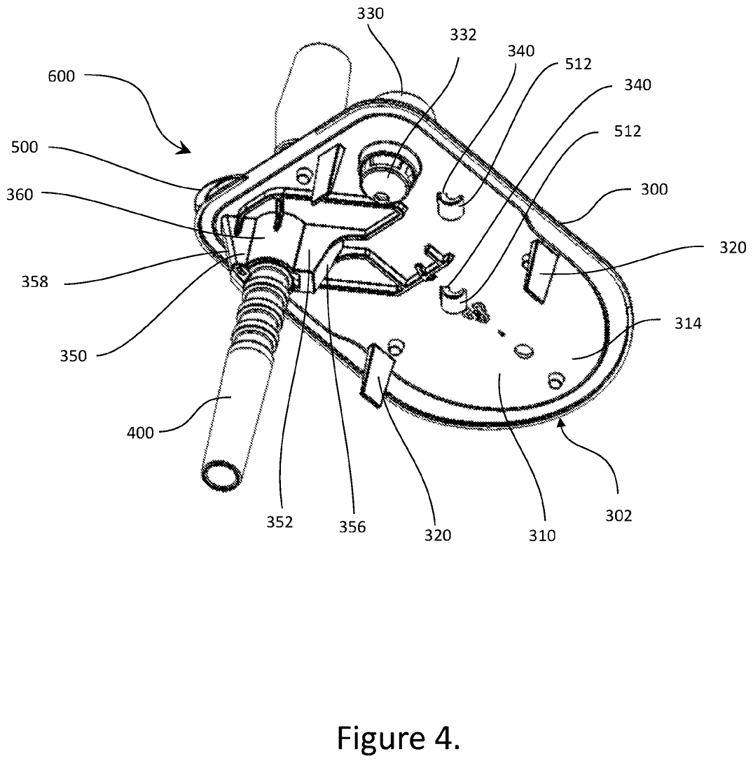

FIGS. 3 and 4 depict an example embodiment of the device or apparatus 600 for retaining the pressure control tube 400 at a user-defined position within the pressure regulator device 200. The apparatus 600 comprises the releasable tube retaining means configured to couple or engage with the top wall 300 of the chamber 210 (not shown).

### Top Wall

The top wall 300 of the chamber 210 may cover or substantially enclose the tank or chamber 210. The top wall 300 may be integrally formed or permanently joined with the side wall or walls 214 of the chamber 210. In other embodiments the top wall 300 may be a separate component that is removably attachable to the chamber 210. In some embodiments the top wall 300 may comprise a lid. The top wall 300 may have a shape that matches an outer perimeter of the chamber 210. The top wall 300 may also cover or substantially enclose an open top of the levelling chamber 220 or overflow facility 230.

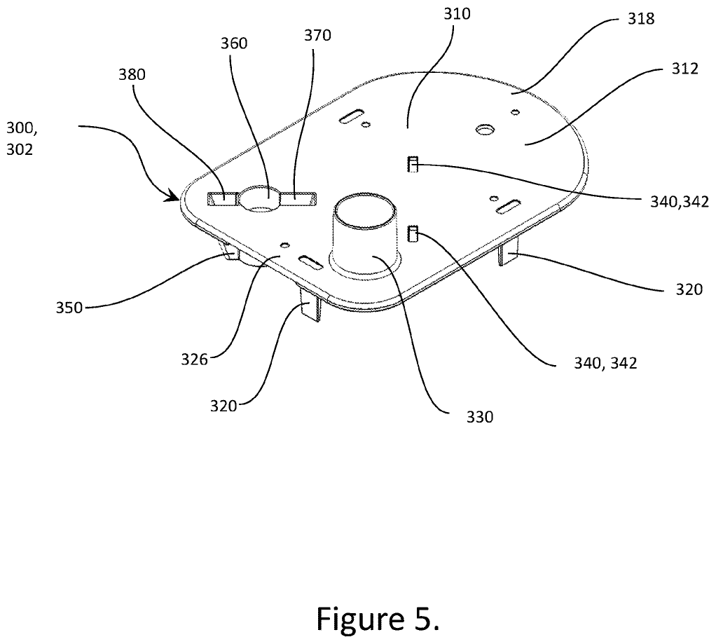

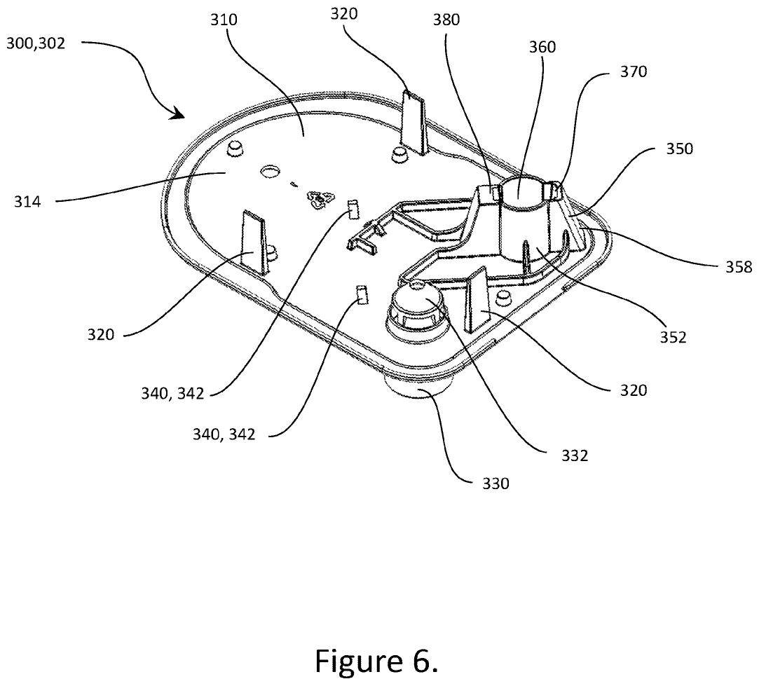

FIGS. 5 and 6 depict an example embodiment of the top wall 300 of the chamber 210. As shown, the top wall 300 comprises a removable cover or lid. The top wall 300 comprises a substantially flat plate, panel, sheet, or wall 310 configured to substantially cover or enclose an open top of the chamber 210. The top wall 300 may comprise a top surface 312 and a bottom surface 314. The top wall 300 may comprise alignment tabs 320 for aligning the lid or top wall 300 with the chamber 210. An outlet port 330 may be provided to the top wall 300 to allow gases to exit the chamber 210. The top wall 300 may comprise a coupling feature 340 for coupling with the releasable tube retaining means 500. The top wall 300 may further comprise a guide portion 350 and a tube aperture 360.

In the illustrated embodiment, the plate or wall 310 that forms the top wall 300 is substantially “D”-shaped. The top wall 300 has three straight sides and one rounded side. The straight sides may form a first end 316. The first end may be substantially square or rectangular and may be configured to cover or enclose the chamber 210. The rounded side may form a second end 318. The second end may be configured to cover or enclose the levelling chamber 220 or the overflow facility 230.

One or more alignment tabs 320 may extend generally downwardly from the bottom surface 314. The alignment tabs may be spaced inwardly from a peripheral edge of the top wall 300 or lid 302. The alignment tabs 320 may be configured to align the lid 302 with an open top of the chamber 210, when assembled. At least one of the alignment tabs may abut or sit adjacent an inner perimeter of the open top of the chamber 210, when the top wall 300 is assembled to the chamber 210. The inner perimeter of the open top of the chamber 210 may be defined by an inner surface of the side wall or walls 214 of the chamber 210. In some embodiments, one or more of the alignment tabs 320 may be positioned inside a perimeter of an open top of the levelling chamber 220.

The outlet port 330 may comprise a short conduit portion which may extend upwardly from the top surface 312. The outlet port 330 may also comprise a shield portion 332 that may extend downwardly or be positioned below the bottom surface 314. The outlet port 330 may be configured to allow gases to exit the chamber 210 to atmosphere. The outlet port 330 can also be used to initially fill the chamber 210 with water or other liquid. The shield 332 may be configured to mitigate risk of liquid aerosols, created by vigorous bubbling on the surface of the water within the chamber 210, being expelled. The outlet port 330 may be located adjacent a corner of the first end 316 of the top wall, such that it is in fluid communication with the chamber 210.

The top wall 300 may comprise a coupling feature 340. The coupling feature 340 may be configured to couple the releasable tube retaining means 500 to the top wall 300. The coupling feature 340 may be configured to be coupled with a corresponding coupling portion 512 (described further below) of the tube retaining means 500. The coupling feature 340 may comprise one or more brackets, posts, hinges, slots or structures suitable for receiving or being coupled to the coupling portion 512 of the tube retaining means 500. The coupling feature 340 may be configured to provide a permanent connection between the top wall 300 and the tube retaining means 500. Alternatively, the coupling feature 340 may be configured to provide a releasable connection between the top wall 300 and the tube retaining means 500.

The coupling feature 340 may comprise one or more slots or recesses 342. In the embodiment illustrated in for example, FIGS. 5 and 6, the coupling feature 340 comprises a pair of spaced apart slots 342. The slots 342 may comprise elongate apertures that extend at least partly through the top wall 300. The pair of slots 342 may be co-linear and extend longitudinally in a diagonal direction relative to the straight sides of the top wall 300. The slots 342 may be in or near a middle region of the top wall 300. The slots 342 may be configured in use to receive at least a part of the coupling portion 512 (described further detail) of the releasable tube retaining means 500, such that the releasable tube retaining means 500 can be coupled to or engaged with the lid 300. In alternative embodiments, the coupling feature 340 may take any other form suitable for coupling the releasable tube retaining means 500 to the top wall 300 or a side wall of the chamber 210.

The tube aperture 360 may comprise an opening that may extend through the top wall 300. The tube aperture 360 may be configured, in use, to receive at least a portion of the pressure control tube 400. The tube aperture 360 may be configured to enable the pressure control tube 400 to extend through the tube aperture 360 and into the body of liquid in the chamber 210. The tube aperture 360 may be circular. The tube aperture 360 may have a diameter that is the same, comparable to, or larger than an outer diameter of the pressure control tube 400. The tube aperture 360 may be located near a corner of the first end 316 of the top wall 300. The tube aperture 360 may be located near a corner opposite the outlet port 330.

The top wall 300 may comprise a guide portion 350. The guide portion 350 may extend generally downwardly from the bottom surface 314. The guide portion 350 may be configured to receive a locking portion or member 540, 560 (described further below) of the releasable tube retaining means 500. The guide portion 350 may be configured to guide the or a locking member 540, 560 into alignment, contact or engagement with the pressure control tube 400, in use.

The guide portion 350 may comprise one or more walls 352, 356, 358 that may extend downwardly from the bottom surface 314 of the top wall 300. The guide portion 350 may partially surround or be positioned adjacent to the tube aperture 360. The walls 352, 356, 358 may be configured to constrict lateral movement of a locking member 540, 560 of the tube retaining means 500. At least one of the one or more of the walls 352, 356, 358 may be angled towards a central axis 390 of the tube aperture 360. The angled wall may be configured to guide locking member 540, 560 into contact or engagement with the pressure control tube 400 when the locking member 540, 560 is inserted into the guide portion 350. The guide portion 350 may comprise a pair of opposing side walls 352 and a pair of opposing end walls 356, 358.

The guide portion 350 may comprise a tube guide portion 362, configured to receive the pressure control tube 400. The tube guide portion 362 may comprise one or more walls that are at least partly cylindrical. The tube guide portion 362 may be coaxial with the tube aperture 360. The wall of the tube guide portion 362 may extend downwardly from the bottom surface 314. The tube guide portion 362 may be formed at least partially by the side walls 352 of the guide portion 350. The tube guide portion 362 may comprise a shaft or tube. The tube guide portion 362 may be configured to restrict movement of the pressure control tube 400 to an axial direction, along the axis 390. The tube guide portion 362 may be configured to guide the pressure control tube 400 to be movable in a substantially vertical or up and down direction within the pressure regulator device 200.

The walls 352, 256, 358 of the guide portion 350 may be configured to form one or more cavities or pockets 370, 380. The one or more pockets 370, 380 may each be configured to receive a locking member 540, 560 of the tube retaining means. The one or more pockets 370, 380 may be positioned adjacent the tube guide portion 362.

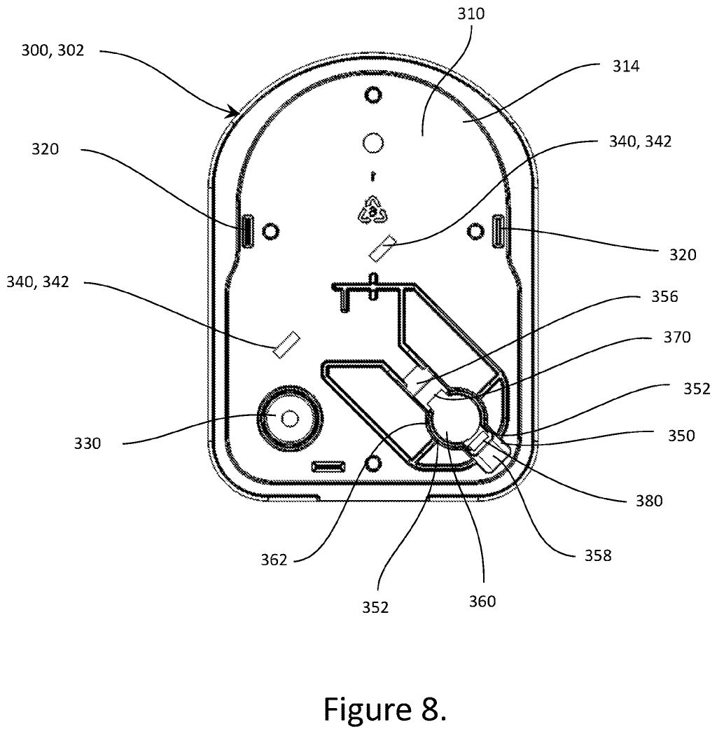

An example embodiment of the guide portion 350 is shown in FIGS. 6 to 9. The guide portion 350 may extend from the bottom surface 314 of the top wall 300. The guide portion 350 may comprise a pair of opposing side walls 352 and a pair of opposing end walls 356, 358. The side walls 352 may be longer than the end walls 356, 358. The side walls 352 and end walls 356, 358 may be configured to form one or more pockets 370, 380 and at least part of the tube guide portion 362.

As illustrated, the tube guide portion 362 may comprise a part-cylindrical wall. The part-cylindrical wall is formed, at least in part by the side walls 352. In other words, the side walls 352 comprise part-cylindrical portions that are configured to form the tube guide portion 362. The cylindrical wall of the tube guide portion 362 may also comprise a bridging portion 364 that extends between the side walls 352. The tube guide portion 362 may comprise a shaft or passage configured to receive the pressure control tube 400 in use, as shown in FIG. 4.

In the embodiment shown, the guide portion 350 comprises a pair of pockets 370, 380. The pair of pockets 370, 380 may be arranged at or adjacent opposing sides of the tube guide portion 360. The pair of pockets 370, 380 may comprise a first pocket 370 and second pocket 380. The first pocket 370 and second pocket 380 may comprise hollow pockets or cavities within the guide portion 350 that may be diametrically opposed about the tube guide portion 360. The first pocket 370 may be configured to receive a first locking member 540 of the releasable tube retaining means 500 (described further below), in use. The second pocket 380 may be configured to receive a second locking member 540 of the releasable tube retaining means 500 (described further below) in use.

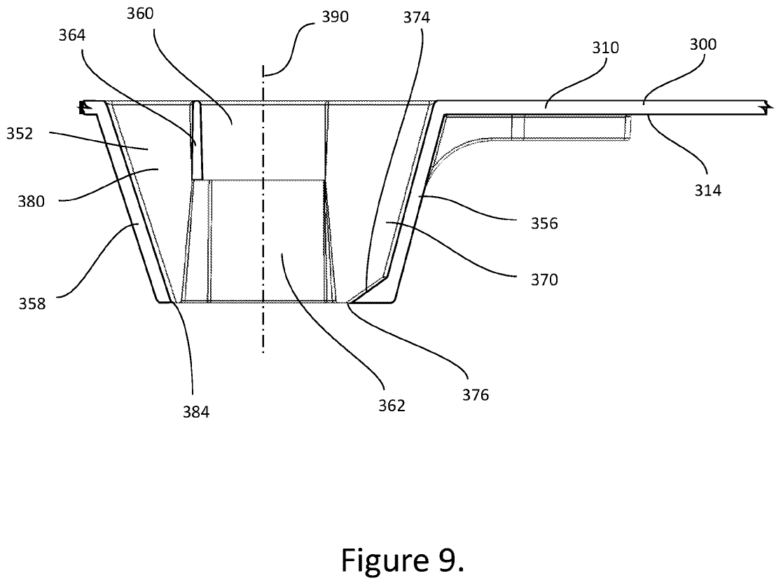

In the embodiment shown in FIG. 9, each of the first and second pockets 370, 380 are formed by side walls 352 and end walls 356, 358. The first pocket 370 is formed by a first end wall 356 and a portion of each of the opposing side walls 352. The first end wall 356 may extend at an angle from the bottom surface 314 towards the central axis 390 of the tube aperture 360 and tube guide portion 362. Portions of the side walls 352 that form the first pocket 370 may extend substantially vertically from the bottom surface 314.

The second pocket 380 may be formed by a second end wall 358 and a portion of each of the opposing side walls 352. The second end wall 358 may extend at an angle from the bottom surface 314 towards a central axis 390 of the tube aperture 360 tube guide portion 362. The portions of the side walls 352 that form the second pocket 380 may extend substantially vertically from the bottom surface 314.

The first end wall 356 may comprise a ramp surface 374 at a distal end relative to the bottom surface 314. The ramp surface 374 may be configured to be selectively or releasably engageable with a locking member 540 of the tube retaining means 500, in use. The ramp surface 374 may be angled relative to a proximal end and/or main body portion of the first end wall 356 and the central axis 390 of the tube guide portion 360. The angle between the ramp surface 374 and the central axis 390 may be greater than the angle between the proximal end and/or main body portion of the first end wall 356 and the central axis 390.

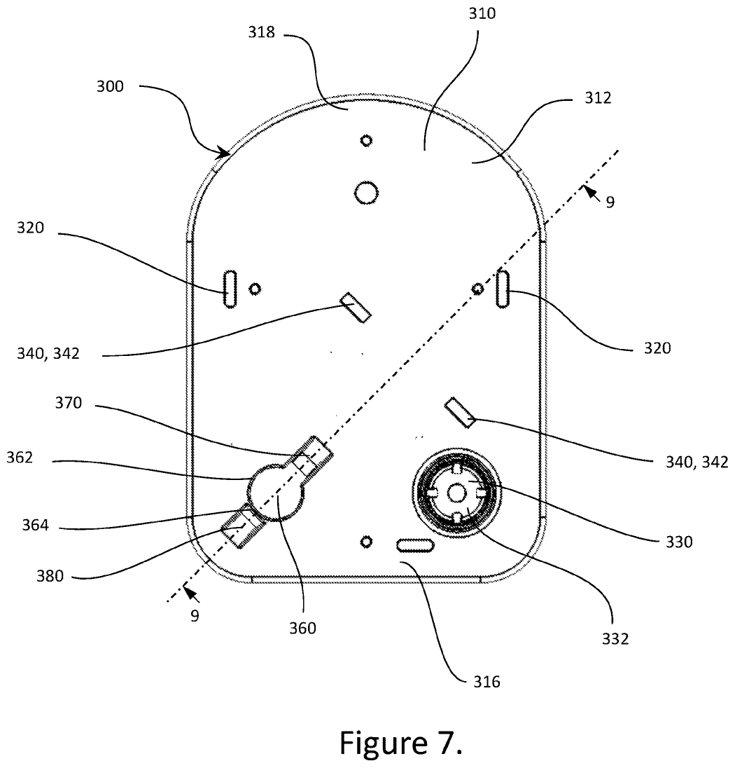

The first and second pockets 370, 380 may be open at a bottom, lower or distal end, such that in use, a lower edge 376 of the ramp surface 374 and a lower edge 384 of the second end wall 358 are spaced apart from the pressure control tube 400. As shown in FIGS. 7 and 9, the first pocket 370 and the tube guide portion 362 may comprise a single or continuous opening or cavity. The second pocket 380 may be at least partially separated from the tube guide portion 362 by the bridge portion 364.

### Pressure Control Tube

The pressure control tube 400 may comprise an elongate tube or conduit. The pressure control tube 400 may comprise or be attachable to, an end portion of an expiratory conduit 130 of a breathing apparatus. The pressure control tube 400 may be configured to be selectively moveable within the column or body of liquid within the chamber 210. Raising or lowering the pressure control tube 400 within the body of liquid may increase or decrease a depth of submersion of the pressure control tube 400 within the body of liquid. A depth of submersion of the pressure control tube 400 within the body of liquid may determine a pressure of gases delivered to a patient by the pressure regulator device 200.

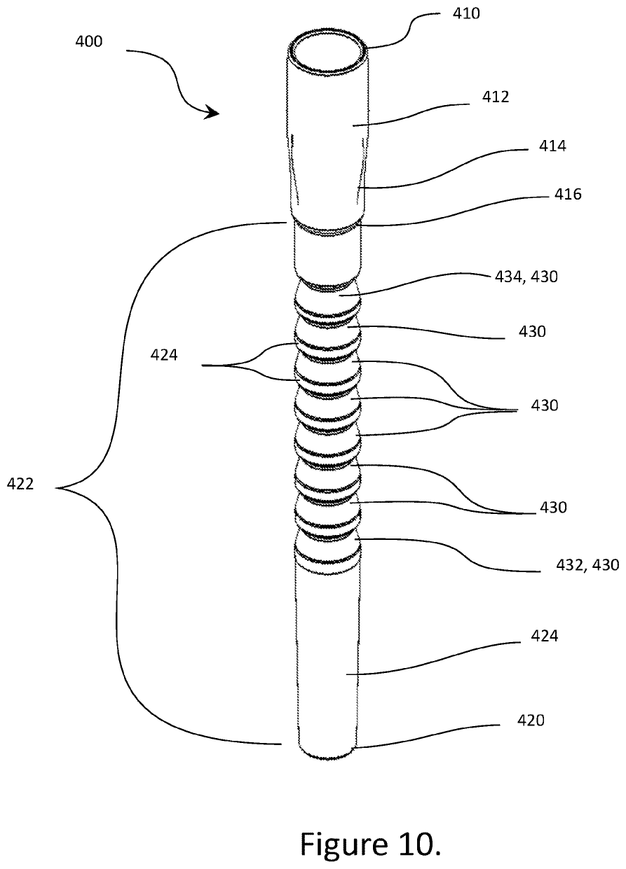

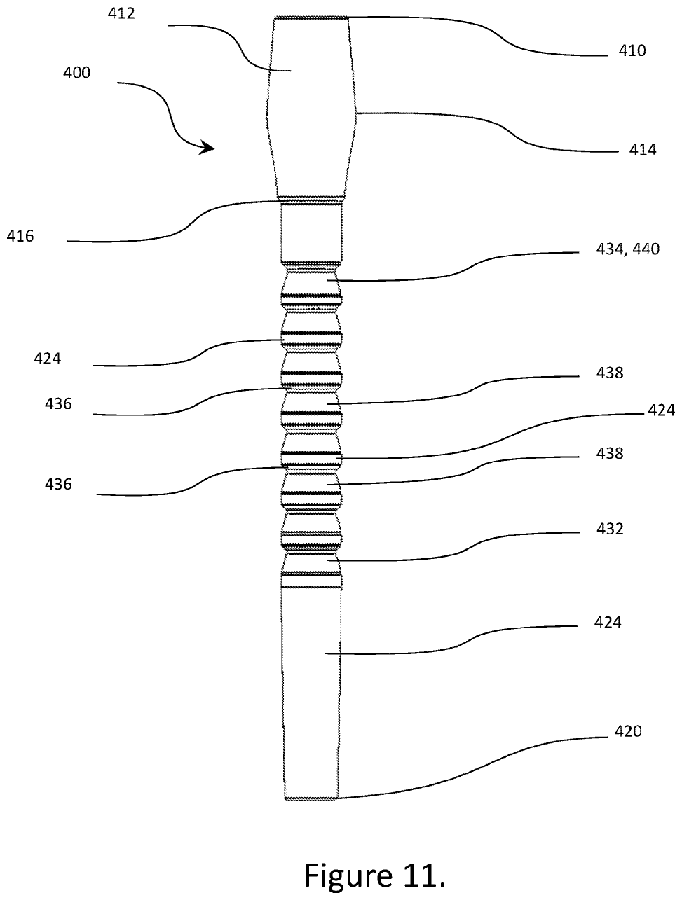

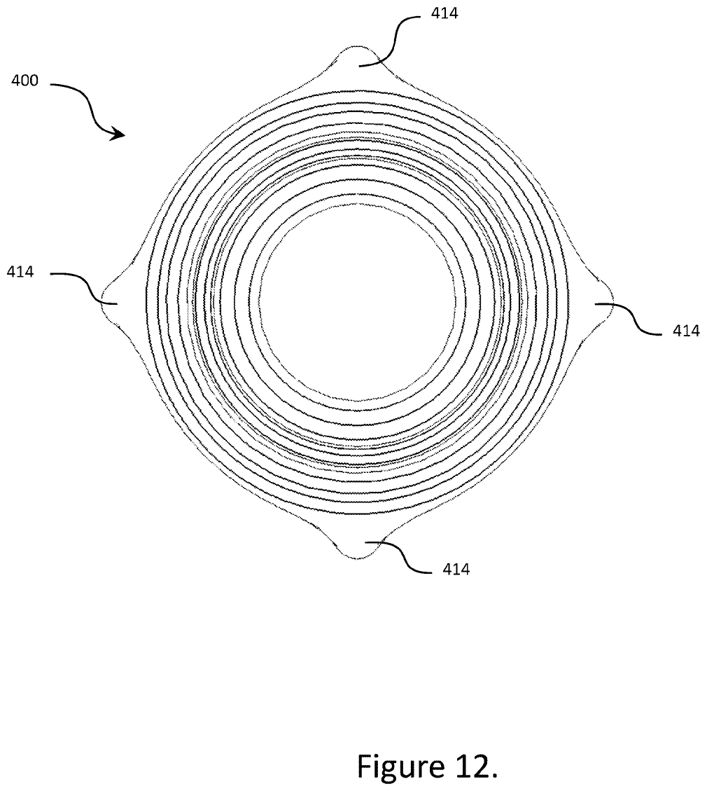

As shown in FIGS. 10 to 13, the pressure control tube or probe 400 may comprise a substantially rigid elongate tube or conduit. The pressure control tube 400 may comprise a first end or upper end 410, a second end or lower end 420 and a shaft or body portion 422 extending therebetween. A grip portion 412 may be positioned at or adjacent the upper end 412. The pressure control tube 400 may also comprise one or more engagement features 430 that may be configured to be selectively engageable with one or more locking members 540, 560 of the releasable tube retaining means 500.

The upper end 410 may be configured to be fluidly connected to an end of an expiratory tube 130. Alternatively, the pressure control tube 400 may form a part of an expiratory tube 130. The lower end 420 is configured to be in fluid communication with the body or column of water within the chamber 210, when the pressure regulator device 200 is in use. The depth or submersion level of the lower end 420 within the column of water determines the mean pressure of the gases delivered by the pressure regulator 200.

The grip portion 412 may comprise a bulbous or protruding portion of the shaft or body 422 of the pressure control tube 400. At least a part of the grip portion 412 may have a greater diameter or width than the shaft of the body 422. The pressure control tube 400 may have a lip or step 416 where the grip portion 412 ends and the body 422 begins. The step or lip may form a lower or bottom surface of the grip portion 412. The grip portion 412 may be configured to be positioned above the top wall 300, in use. The grip portion 412 may be configured to be gripped by a user to adjust a position of the pressure control tube 400 within the pressure regulator device 200. A user may hold onto or grip the grip portion and apply downwards or upwards force to raise or lower the pressure control tube 400 within the chamber 210.

An outer surface 424 of the body portion or shaft 422 of the pressure control tube 400 may be cylindrical. The outer surface 424 may have a substantially constant diameter between each of the engagement features 430. The outer surface 424 can taper to a smaller diameter between a lowermost engagement feature 432 and the lower end 420. In alternative embodiments, the body of shaft 422 may have any suitable cross-sectional shape. For example, the cross-section of the body or shaft may be triangular, square, hexagonal or any other polygonal shape.

One or more engagement features 430 may be located on the outer surface 424. The pressure control tube 400 may comprise a plurality of engagement features. The plurality of engagement features may be spaced along at least part of a length of the body 422. Each of the plurality of engagement features 430 may be uniformly spaced apart. In alternative embodiments, the pressure control tube 400 may include one or more elongate ribs. The ribs may protrude outwardly from the outer surface 424 and longitudinally along a length of the body or shaft 422. A plurality of engagement features may be located along a length of the one or more ribs.

Each of the one or more engagement features 430 may comprise a notch, groove or indent that protrudes inwardly from the outer surface 424 of the body 422. The engagement features 430 may be annular, circumferential, or perimetrical. The engagement features 430 may be part-annular or partially circumferential. In some embodiments the engagement features 430 may extend around a part or portion of the circumference or perimeter of the pressure control tube 400. In some embodiments the engagement features 430 may comprise a plurality of non-continuous notches or grooves spaced about the circumference of the pressure control tube 400. In some embodiments the engagement features 430 may comprise pairs of diametrically opposed notches, grooves, or indents.

The engagement features 430 of the pressure control tube 400 may be configured in use to be engageable with at least a portion of a locking member 540, 560 of the tube retaining means 500. The engagement features 430 may provide one or more discrete positions or settings at which the pressure control tube 400 can be selectively engageable with the releasable tube retaining means 500. The engagement features 430 may correspond to a plurality of discrete user-defined positions at which the pressure control tube 400 can be selectively positioned within the pressure regulator device 200. The engagement features 430 may correspond to a plurality of discrete heights within the chamber 210 at which the pressure control tube 400 can be selectively positioned by a user. The engagement features 430 may correspond to a plurality of discrete user-defined depths of submersion within the body of liquid in which the pressure control tube 400 can be selectively positioned by a user. Each of the one or more engagement features 430 may correspond to a discrete pressure setting of the pressure regulator device.

As shown in FIGS. 10 to 13, the pressure control tube 400 comprises a plurality of engagement features 430, spaced uniformly along a portion of the body 422. In the embodiment shown there are eight engagement features 430, though other number of engagement features 430 could be provided. Each of the engagement features 430 corresponds to a predetermined pressure setting of the pressure regulator device 200.

The engagement features 430 of the illustrated embodiments comprise a plurality of circumferential or annular notches, indents, or grooves. The engagement features 430 are configured to be selectively engageable with the locking members 540, 560 of the releasable tube retaining means 500, as described in further detail below.

The spacing of the engagement features 430 along the length of the body portion 422 may determine a change in depth of submersion of the lower end 420 of the pressure control tube 400 within the body of liquid. The change in depth of submersion of the lower end 420 determines the change in pressure of the pressure regulator device 200 between each of the predetermined pressure settings. In the illustrated embodiment each of the engagement features 430 are spaced apart by a distance of 1 cm, which corresponds to a pressure change of 1 cmH₂O. The eight engagement features 430 are configured to provide predetermined pressure settings between 3 cmH₂O and 10 cmH₂O.

In alternative embodiments, smaller spacings between the engagement features 430 may provide smaller increments in pressure settings. For example, a spacing of 5 mm may provide a pressure change or 0.5 cm H₂O. In some embodiments smaller or larger spacings between engagement features 430 may provide a corresponding smaller or larger change in pressure setting.

A first or lowermost engagement feature 432 may be offset from the lower end 420. The lowermost engagement feature 432 may be offset by a distance that may be configured to provide the desired first pressure setting (in this case 3 cm H₂O) when the chamber is filled to a predetermined level with water. A last or uppermost engagement feature 434 may be offset from the grip portion 412 by a distance that may substantially correspond to a length of the first locking member 540.

Each of the engagement features 430 may have a cross-section configured to receive at least a portion of a tube engagement portion 542, 562 of the locking members 540, 560 of the releasable tube retaining means 500.

In the illustrated embodiment, each of the engagement features 430 have a substantially triangular cross-section. Each of the engagement features 430 may comprise a first or upper surface 436 and a second or lower surface 438. The upper surface 436 and lower surface 438 may be radially recessed, indented or offset into the body portion 422 relative to the outer surface 424. The upper surface 436 and lower surface 438 may be angled relative to each other and the outer surface 424. The upper and lower surface 436 and 438 may intersect with each other at a corner 442.

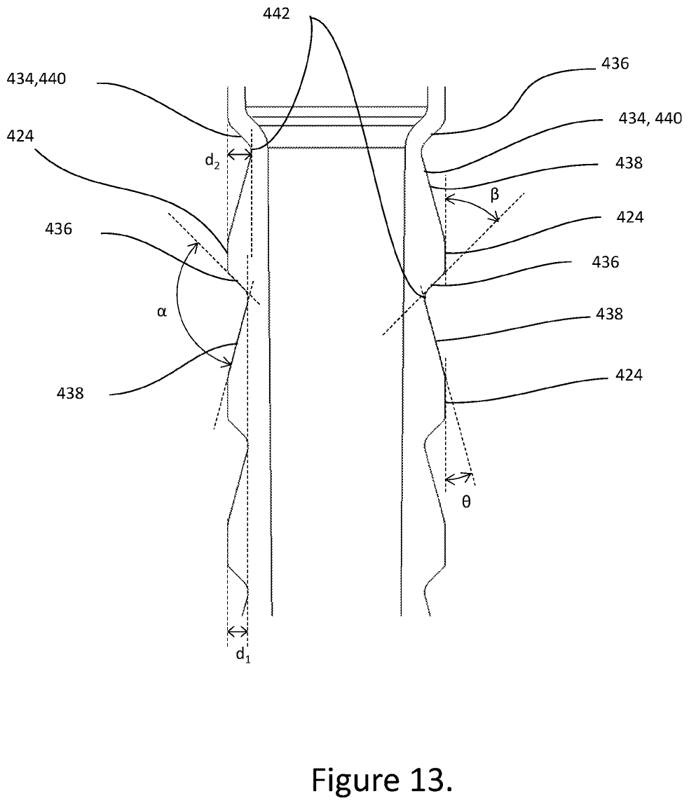

As shown in FIG. 13, an internal angle α may be defined between the upper surface 436 and the lower surface 438 at the corner 442. An external angle β may be defined between the upper surface 436 and the outer surface 424. An external angle θ may be defined between the lower surface 438 and the outer surface 424. The angle α may be greater than 90°. In the illustrated embodiment the angle α is 120°. The angle β may be less than 90°. In the illustrated embodiment the angle β is 45°. The angle θ may be less than 90°. In the illustrated embodiment the angle θ is 15°. The angle β may be greater than 0. In alternative embodiments the angles α, β and θ may have any suitable value.

The angles β and θ may contribute to the level of resistance to movement of the pressure control tube 400 within the pressure regulator device 200. The greater the angle the greater the resistance to movement of the pressure control tube 400 may be. It may be desirable for angle β to be greater than angle θ. When angle β is greater than angle θ, resistance to downward movement of the pressure control tube 400 may be greater than resistance to upwards movement of the pressure control tube 400. Having a greater resistance to downwards movement than upwards movement may assist to mitigate risk of unintentional increases in the pressure setting of the pressure regulator device 200.

The corner 442 at the intersection of the upper surface 436 and lower surface 438 may comprise a rounded surface. The corner 442 may define an innermost point of each of the engagement features 430. The corner 442 may define a depth d1 of the engagement features 430, as shown in FIG. 13.

The pressure control tube may comprise a recessed feature 440. The recessed feature 440 may be configured to enable the tube retaining means 500 to be selectively engageable with the pressure control tube 400 in at least one position in which the releasable tube retaining means 500 is in a substantially unstressed state. The recessed feature 440 may be configured to receive at least a portion of a locking member 540, 560 of the tube retaining means 500. The recessed feature 424 may comprise a notch, groove or indent that extends inwardly from an outer surface 424 of the body 422. The recessed feature 440 may have a depth d2 between an innermost point and the outer surface 424 of the pressure control tube 400. The depth d2 may be such that when a locking member 540, 560 of the tube retaining means 500 is selectively engaged with the recessed feature 440, a portion of the locking member 540, 560 is received in the recessed feature 440. The portion of the locking member 540, 560 received in the recessed feature 440 may be at least partially spaced away from a surface of the recessed feature 440.

In the illustrated embodiment, the recessed feature 440 comprises the uppermost engagement feature 434. In alternative embodiments, the recessed feature 440 may comprise a feature that is independent from the engagement features 430. As shown in FIG. 13, the recessed feature 440 comprises an upper surface 436 and lower surface 438 having the same angles α, β, and θ as the other engagement features 430. The recessed feature 440 may be recessed or offset relative to the outer surface 424 in comparison to the other engagement features 430. The recessed feature 440 has a depth d2 defined by the distance between the corner 442 where the upper and lower surfaces 436, 438 intersect and the outer surface 424. The depth d2 of the recessed feature may be greater than the depth d1 of the rest of the engagement features. In the illustrated embodiment the depth d2 is 0.2 mm greater than d1. However, it is to be understood that other differences in depth may also be suitable.

### Releasable Tube Retaining Means

The releasable tube retaining means 500 may be configured to releasably retain, secure, or hold the pressure control tube 400 in a user-defined position within the pressure regulator device 200. The tube retaining means 500 may be configured to retain the pressure control tube in a user-defined position relative to a wall of the chamber 210. The releasable tube retaining means 500 may be configured to be engageable with or coupled to a wall of the chamber 210. The tube retaining means 500 may be configured to be coupled to the top wall 300 or lid 302. The tube retaining means 500 may be configured to retain the pressure control tube 400 in one or more user-defined positions that correspond to one or more pressure settings of the pressure regulator device 200.

The tube retaining means 500 may be configured to resist movement of the pressure control tube 400 within the pressure regulator device 200.

The tube retaining means 500 may comprise a resiliently deformable component or device. At least a portion of the tube retaining means 500 may be configured to be releasably engageable with the pressure control tube 400. At least a portion of the tube retaining means 500 may be configured to be engageable with an engagement feature 430 of the pressure control tube 400. The releasable tube retaining means 500 may alternatively be referred to a locking clip or a lock.

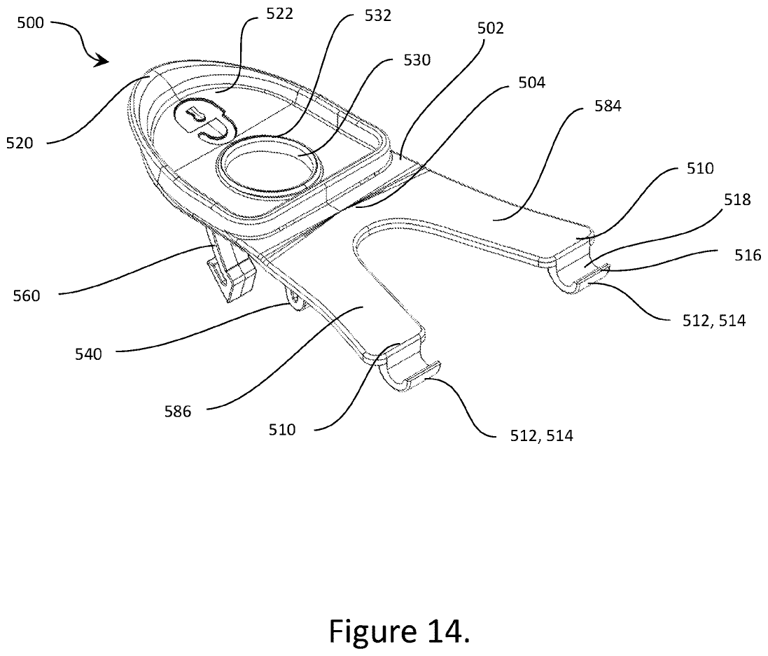

FIGS. 14 to 17 show an example embodiment of the releasable tube retaining means 500 in isolation. The releasable tube retaining means 500 may be made from a resiliently deformable material such as a metal or plastic. The releasable tube retaining means 500 may comprise an elongate body 502 having an upper or top surface 504 and an opposing lower or bottom surface 506. The upper surface 504 may be configured, in use, to face upwardly away from the top surface 312 of the top wall 300. The lower surface 506 may be configured, in use, to face or at least partially abut the top surface 312 of the top wall 300. The elongate body 502 may comprise a first end 510, and an opposing second end 520. A hinge/fulcrum 508 and a tube receiving portion 530 may be provided intermediate the first and second ends 510, 520. The elongate body 502 may further comprise a resiliently deformable region 580. The resiliently deformable region 580 may extend from the first end 510 towards the hinge/fulcrum 508.

The tube retaining means may further comprise a first locking member 540 and an opposing second locking member 560. The first and second locking members 540, 560 may be configured to be selectively and/or releasably engageable with the engagement features 430 of the pressure control tube 400. When selectively engaged with an engagement feature 430, the first and second locking members 540, 560 may be configured to retain or hold the pressure control tube 400 in a user-defined position within the pressure regulator device 200.

The first end 510 of the elongate body 502 of the releasable tube retaining means 500 may comprise a coupling end. The first end 510 may be configured to be coupled to or otherwise engaged with, a wall of the chamber 210. In the illustrated embodiment the first end 510 is configured to be coupled to the top wall 300. The first end 510 may comprise one or more coupling portions 512. The coupling portions 512 may be configured to be coupled with the one or more coupling features 340 of the top wall 300. The coupling portions 512 may be configured to be releasably coupled to the coupling features 340. The coupling portions 512 may be pivotably coupled to the coupling features 340.

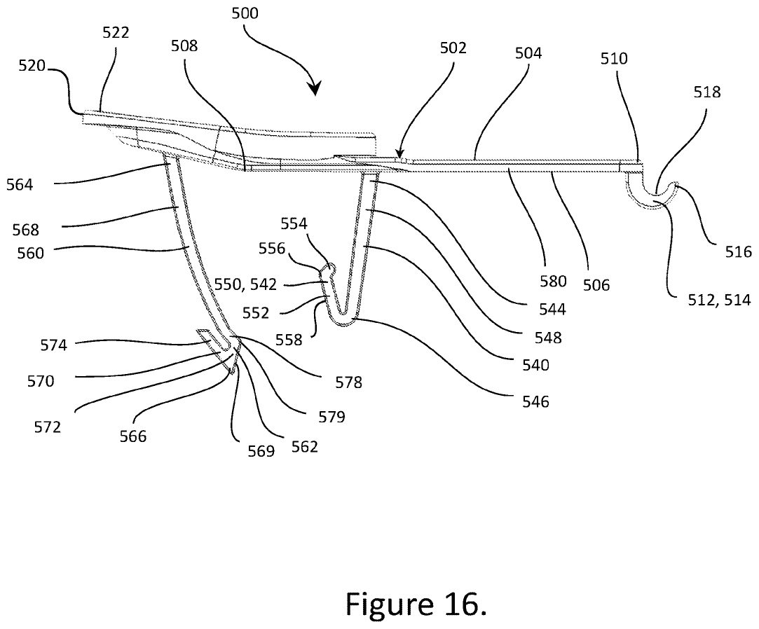

The coupling portions 512 may comprise a tongue, tab, or hook portion configured to be received by or connected to the coupling features 340. The coupling portions 512 may extend from the first end 510. In the illustrated embodiment, the first end 510 comprises a pair of coupling portions 512. The coupling portions 512 each comprise a hinge hook 514. As shown in FIG. 16, the hinge hooks 514 may comprise part-circular or substantially sickle-shaped hook portions. The hinge hooks 514 may initially extend downwardly from the lower surface 504 and then curve upwardly away from the first end 510 of the elongate body 502.

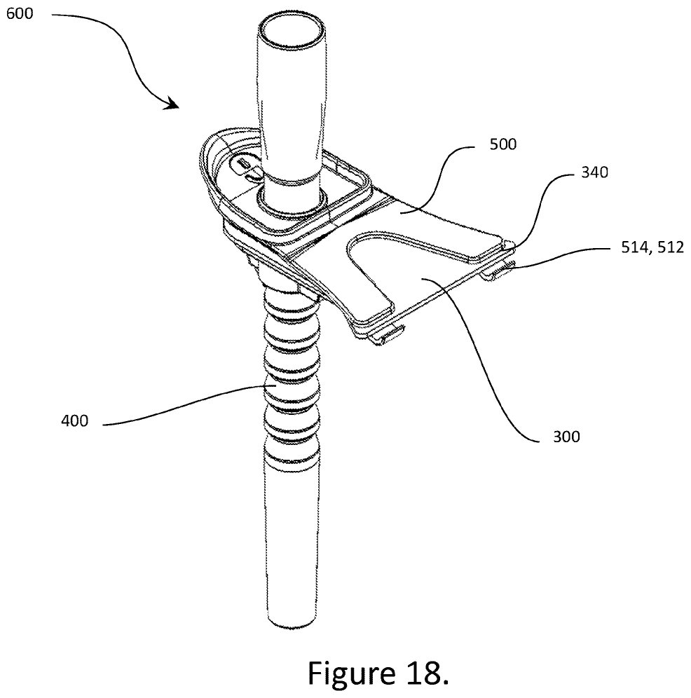

The hinge hooks 514 may be configured to be inserted into the slots of the clip retaining means 340, as shown in FIG. 4 or 18. The hinge hooks 514 may have a terminal end 516 that may be positioned adjacent or may abut with the bottom surface 314 of the top wall 300 in use. This may retain the first end 510 in engagement with the top wall 300, which may minimise upward movement of the first end 510 when the resiliently deformable region 580 is deformed. A curved inner surface 518 of the hinge hooks 514 may be configured to engage with an edge of the engagement features 340. When the engagement features 430 comprise slots that extend through the top wall 300, the inner surface 518 may abut with and/or engage with an edge of the slots, in use. The hinge hooks 514 can engage with the coupling features 340 such that the releasable tube retaining means 500 is pivotably coupled to the top wall 300. The pivotal coupling may enable the releasable tube retaining means 500 to pivot or rotate about its first end 510, relative to the top wall 300, when the pressure control tube 400 is not inserted through the tube receiving portion 530.

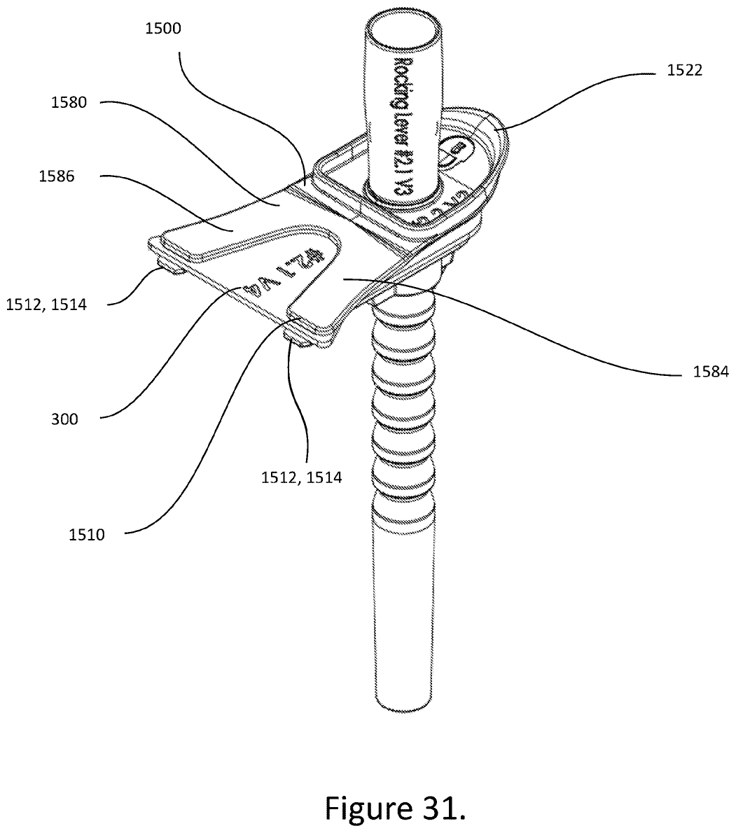

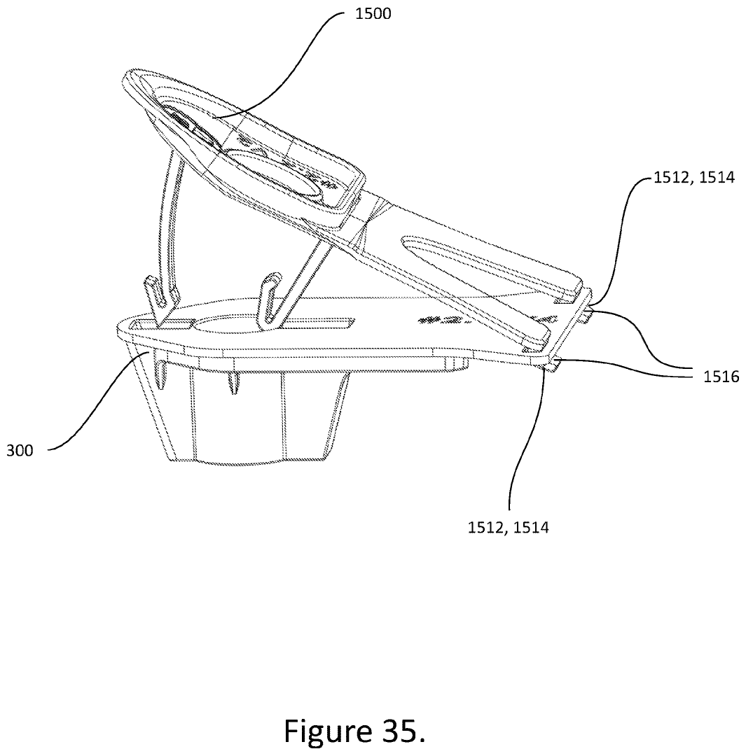

FIGS. 31 to 35 show an alternative embodiment of a tube retaining means 1500 including an alternative embodiment of coupling portions 512. Tube retaining means 1500 can be substantially the same as the tube retaining means 500 except as described below. Thus, any features not specifically described can be substantially the same as or similar to corresponding features of the tube retaining means 500 or other tube retaining means described herein or can be of another suitable arrangement.

Releasable tube retaining means 1500 may comprise one or more coupling portions 1512. The coupling portions 1512 may be located at a first end 1510 of the tube retaining means 1500. The coupling portions 1512 may be configured to couple the tube retaining means 1500 to the top wall 300 of the pressure regulator device 200. The coupling portions 1512 may be configured to be coupled with the one or more coupling features 340 of the top wall 300. The coupling portions 1512 may be configured to be releasably coupled to the coupling features 340.

The coupling portions 1512 may comprise one or more tongues or tabs configured to be received by or connected to the coupling features 340. The coupling portions 512 may extend from the first end 510. In the illustrated embodiment, the releasable tube retaining means 1500 comprises a pair of coupling portions 1512 that are spaced apart and located at ends of elongate spring arms 1584, 1586. The coupling portions 1512 may each comprise a tongue or tab 1514.

The tongues or tabs 1514 may comprise a first portion that extends generally downwardly from the lower surface 1504. A second portion may extend at an angle to the first portion. The second portion may extend generally parallel to the elongate body 1502. In the embodiment shown in FIGS. 31 to 35, the tongues or tabs 1514 may comprise substantially L-shaped protrusions that extend from a lower surface 1504 of an elongate body 1502 of the tube retaining means 500. The first portion of the tongues or tabs 1514 may extend in a first direction that may be substantially perpendicular to the lower surface 1504. The tabs 1514 may then bend such that the second portion may extend in a second direction that may be substantially parallel to the lower surface 1504. In other words, at least a portion of each of the tabs 1514 may extend laterally from the first end 1510.

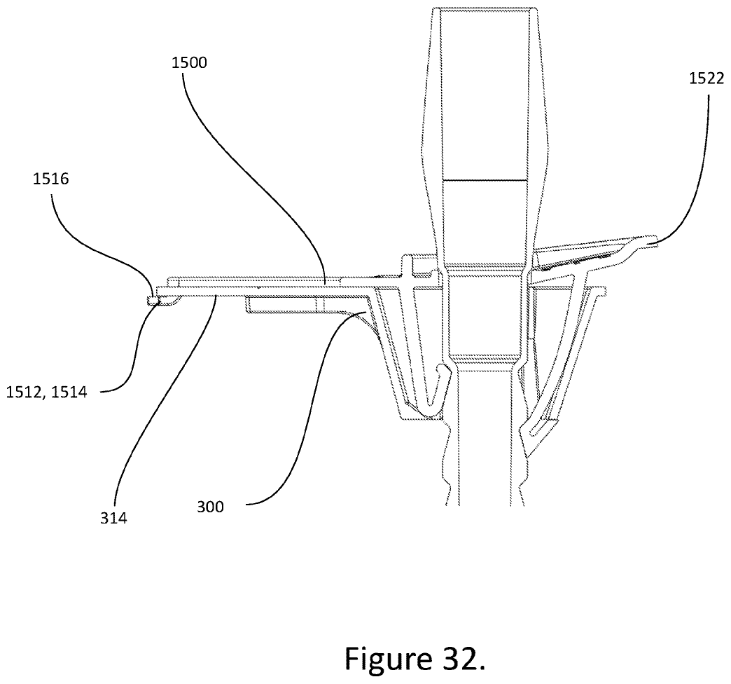

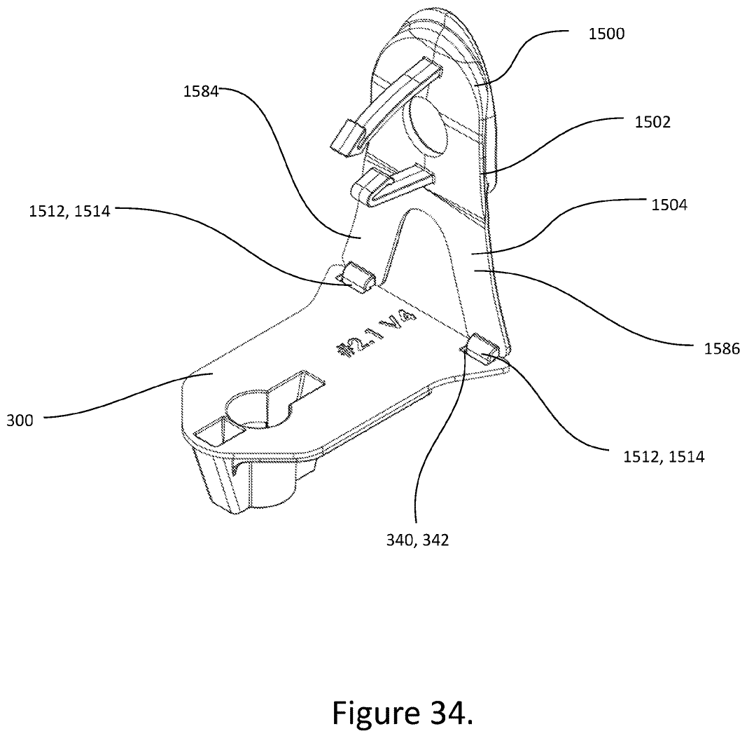

The tabs 1514 may be configured to be inserted into or through the slots 342 of the clip retaining means 340, as shown in FIG. 34. As shown in FIG. 32, the tabs 1514 may have an upper surface 1516 that may be positioned adjacent or against the bottom surface 314 of the top wall 300 in use. The upper surface 1516 may be located on the second portion of the tabs 1514. Contact or engagement between the upper surface 1516 of the tabs 1514 and the bottom surface 314 may resist upwards movement of the first end 1510 of the tube retaining means 1500, when a resiliently deformable region 1580 of the tube retaining means 1500 is deformed in use. This may retain the first end 1510 in engagement with the top wall 300. The tabs 1514 can engage with the coupling features 340 such that the releasable tube retaining means 1500 is pivotably coupled to the top wall 300. The pivotal coupling may enable the releasable tube retaining means 1500 to pivot or rotate about its first end 1510, relative to the top wall 300.

In further alternative embodiments, the releasable tube retaining means 500 may be coupled to the lid 302 in any other suitable manner. For example the first end 510 may comprise a coupling portion 512 in the form of a tab or other feature that is slidably, fixedly or otherwise engageable with a corresponding coupling feature 430 of the top wall 300 or a side wall 214 of the chamber 210. In other words, the tube retaining means 500 may not be pivotably coupled to the lid 302.

The resiliently deformable region 580 of the elongate body 502 may comprise a flat spring or flat plate portion of the elongate body 502. The resiliently deformable region 580 may extend from the first end 510 towards the second end 520. The deformable region 580 may terminate at the hinge/fulcrum 508 intermediate the first and second ends 510, 520 of the elongate body 502. Alternatively, the deformable region 580 may terminate at or adjacent the first locking member 540. The deformable region 580 may be configured to act as a spring. The resiliently deformable region 580 may be configured to enable the tube retaining means 500 to be deformable to adapt between a first and a second configuration (described in detail below). The resiliently deformable region 580 may be configured to bias the tube retaining means 500 towards a first configuration. The deformability of the resiliently deformable region 580 may enable the tube retaining means 500 to be releasably engageable with the pressure control tube 400. The resiliently deformable region 580 may have a thickness configured to enable the resiliently deformable region 580 to flex or bend relative to the top wall 300.

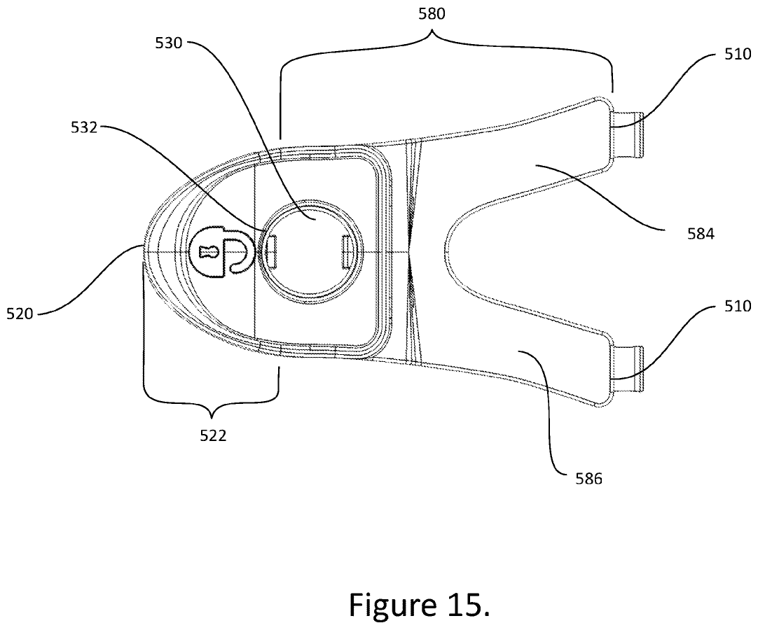

In the embodiment illustrated in for example, FIGS. 15-17, the resiliently deformable region 580 comprises a pair of elongate spring arms 584, 586. As shown in FIG. 15, the spring arms 584, 586 are spaced apart from each other at the first end 510 of the elongate body 502. The spring arms 584, 586 may be angled away from each other towards the first end 510. The spring arms 584, 586 may be formed in a substantially V-shaped arrangement.

In alternative embodiments the releasable tube retaining means 500 may comprise a resiliently deformable region 580 that is a single elongate portion of the elongate body 502, i.e. is not split into two spring arms 584, 586. In such an embodiment there may be a single hinge hook 514 or other coupling portion 512 for coupling the releasable tube retaining means 500 to the top wall 300.

The second end 520 of the elongate body 502 may comprise a free end. The second end 520 may be configured to be moveable relative to the top wall 300. The second end 520 may comprise an actuating member 522. The actuating member 522 may be configured to actuate or adapt the releasably tube retaining means 500 between a first and a second configuration. In some embodiments the actuating member 522 may be of a type referred to as a push tab. The actuating member may be configured to act as a lever.

The actuating member 522 may comprise a portion of the elongate body 502. The actuating member 522 may comprise a tab or flap extending from the elongate body 502. The actuating member 522 may extend at an angle relative to the resiliently deformable region 580 or the elongate body 502, as shown in FIG. 16. The actuating member 522 may extend from the second end 520 towards the first end 510, terminating at the hinge/fulcrum 508. The actuating member 522 may be configured, in use, to adapt the releasable tube retaining means 500 between a first configuration and a second configuration, when urged towards the top wall 300.

The hinge/fulcrum 508 may comprise a corner, edge or ridge extending across a width of the elongate body 502. The hinge/fulcrum 508 may be located intermediate the first and second ends 510 and 520, where the deformable region 580 transitions into the actuating member 522. The hinge/fulcrum 508 may be configured to form a pivot. In use, the actuating member 522 may be configured to selectively pivot about the hinge/fulcrum 508. The resiliently deformable region 580 may be configured to bend or deform between the hinge/fulcrum 508 and the coupling portion 512 when the actuating member 522 is pivoted about the hinge/fulcrum 508. In the illustrated embodiment the hinge/fulcrum 508 comprises a corner on the lower surface 506. The corner may be rounded. The corner of the hinge/fulcrum 508 is located where the actuating member 522 extends at an angle to the remainder of the elongate body 502 or the deformable region 580. The hinge/fulcrum 508 may be offset from a centre of the elongate member 502 towards the second end 520.

The tube receiving portion 530 may be configured to receive the pressure control tube 400. The tube receiving portion 530 may comprise an opening or aperture extending through the elongate body 502. The tube receiving portion 530 may be located intermediate the first end 510 and the second end 520. The tube receiving portion 530 may be located between the resiliently deformable region 580 and the actuating member 522. The tube receiving portion 530 may be located adjacent or overlapping the hinge/fulcrum 508. The tube receiving portion 530 may be configured to align the pressure control tube 400 with the first and second locking members 540 and 560.

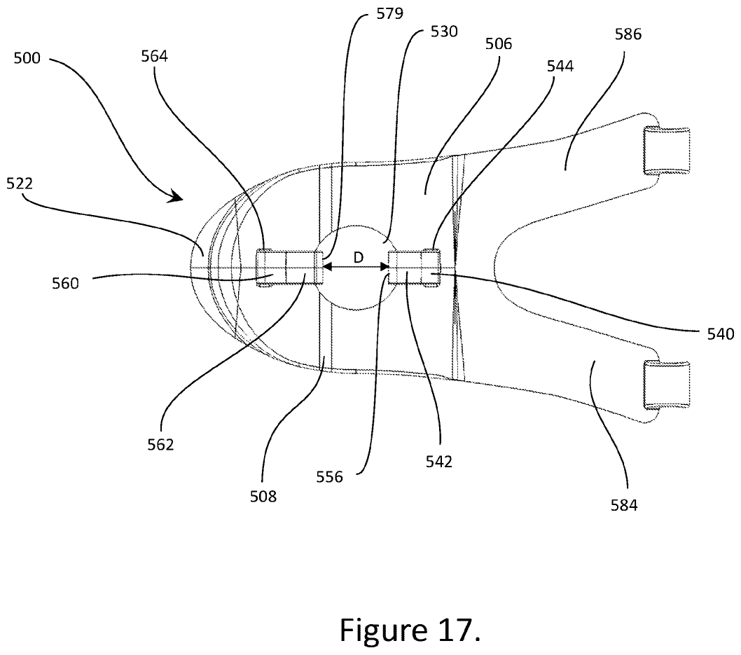

In the illustrated embodiment the tube receiving portion 530 comprises an aperture that extends through the elongate body 502. The aperture is located adjacent the actuating member 522 and extends at least partially through the hinge/fulcrum 508, as shown in FIG. 17. The tube receiving portion 530 may comprise an annular lip or bead 532 that extends around the perimeter of the aperture. The lip or bead 532 may protrude upwardly from the upper surface 504. The bead 532 may be configured to form a stop or limiter that may restrict or limit how far into the chamber 210 the pressure control tube 400 can be lowered. The bead 532 may be configured to abut with the step 416 of the grip portion 412 of the pressure control tube 400, when the pressure control tube 400 is in a fully lowered position. In alternative embodiments the tube receiving portion 530 may comprise a cut-out, notch or slit in a side edge of the tube retaining means 500.

The first and second locking members 540, 560 may be selectively and/or releasably engageable with the engagement features 430 of the pressure control tube 400. The first locking member 540 and second locking member 560 may each comprise elongate legs or members that extend in a generally downward direction from the lower surface 506 of the elongate body 502. The first and second locking members 540, 560 may comprise elongate resiliently deformable members. The first and second locking members 540, 560 may extend downwardly at an angle generally towards each other and the pressure control tube 400 in use. First locking member 540 and second locking member 560 may be diametrically opposed about the tube receiving portion 530, such that in use, they are positioned on opposite sides of the pressure control tube 400.

The first locking member 540 may comprise a tube engagement portion 542. The second locking member 560 may also comprise a tube engagement portion 562. The tube engagement portions 542, 562 may be selectively engageable with one or more of the engagement features 430 of the pressure control tube 400. The tube engagement portions 542, 562 may comprise a protrusion or portion of the first or second locking members 540, 560 respectively. The tube engagement portions 542, 562 may have a surface configured to be a corresponding mating surface with the upper and lower surfaces 436, 438 of the engagement features 430.

In the illustrated embodiment, the first locking member 540, which may also be referred to as a locking leg, comprises a resiliently deformable elongate leg portion 548. The elongate leg portion 548 may be substantially straight. The first locking member 540 may have a proximal end 544 joined to or integrally formed with the elongate body 502. An opposing distal end portion 546 of the first locking member 540 may be a free end. The proximal end 544 may be spaced laterally apart from the tube receiving portion 530 towards the first end 510, as shown in FIG. 17. The tube engagement portion 542 may be positioned at or adjacent the distal end 546. In alternative embodiments the tube engagement portion 542 may be located intermediate the proximal and distal ends 544, 546.

In the illustrated embodiment, the tube engagement portion 542 of the first locking member 540 comprises a hook portion 550. The hook portion 550 may be located at the distal end 544. The tube engagement portion 542 or hook portion 550 may extend generally upwards towards the lower surface 506 and inwardly or towards the pressure control tube 400 in use. The hook portion 550 may comprise a first segment 552 and a second segment 554. The first segment 552 may extend upwardly from the distal end 546 of the leg portion 548 towards the lower surface 506 of the elongate body 502. The first segment 552 may extend at an acute angle relative to the elongate leg 548. The second segment 554 may extend upwardly from an end of the first segment 552 towards the lower surface 506 and towards the leg portion 548. A corner 556 may be formed between the first segment 552 and the second segment 554. The hook portion 550 may be configured to extend a flexural length of the first locking member 540.

The tube engagement portion 542 of the first locking member 540 may comprise a first contact surface 558. The first contact surface 558 may be configured to be a corresponding mating surface with the upper and lower surface 436, 438 of the engagement features 430. The first contact surface 558 may be configured to be selectively engageable with at least one engagement feature 430.

The first contact surface may be provided on a pressure control tube 400 facing side of the hook portion 550. The first contact surface 558 may be formed by the first segment 552, corner 556, and second segment 554 of the hook portion 550. The first contact surface 558 may be a mirror image of the upper and lower surfaces 436, 438 of the engagement features 430. The corner 556 may form part of the first contact surface 558. The corner 556 may define an angle the same as or comparable to angle α of the engagement features 430. The contact surface 558 may be configured to be selectively contactable, mateable and/or engageable with the upper surface 436 and lower surface 438 of the engagement features 430.

The second locking member 560, which may also be referred to as a catch-detent leg, may comprise a resiliently deformable elongate leg portion 568. The elongate leg portion 568 may be curved. The elongate leg portion 568 may be concave towards the lower surface 506. The second locking member 560 may have a proximal end 564 joined to or integrally formed with the elongate body 502. An opposing distal end 566 may be a free end. The proximal end 564 may be spaced laterally apart from the tube receiving portion 530 towards the second end 520. The second locking member 560 may extend downwardly from the lower surface 506 of the actuating member 522. The tube engagement portion 562 of the second locking member 560 may be located at or adjacent the distal end 566. In alternative embodiments, the tube engagement portion 562 may be located intermediate the proximal and distal ends 564, 566.

In the illustrated embodiment, a lower portion 578 of the elongate leg portion 568 is substantially linear relative to the curvature of leg portion 568. The second locking member 560 may comprise a hook portion 570 that extends outwardly or away from the pressure control tube in use. The hook portion 570 may extend from the distal end 566. The hook portion 570 may comprise a first segment 572 and a second segment 574. The first segment 572 may extend generally downwards at an angle from the lower portion 578 of the leg portion 568 and away from the lower surface 506 of the elongate body 502. A corner 579 may be formed between the lower portion 578 and the first segment 572. The second segment 574 may extend upwardly from an end of the first segment 572, towards the lower surface 506 and away from or parallel with the leg portion 568.

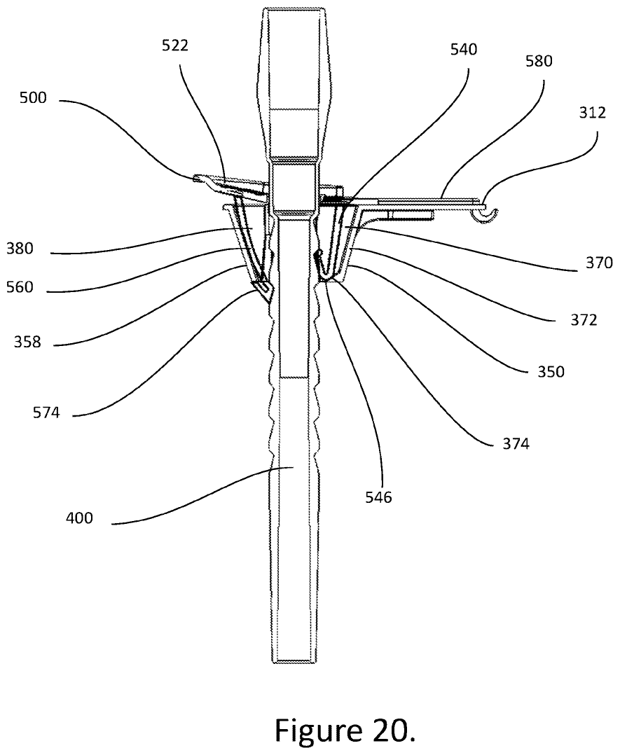

At least part of the hook portion 570 may be configured to act as a catch. The second segment 574 may be configured to form a catch. The catch may be configured to engage with a portion of the top wall 300. As shown in FIG. 20, the second segment 574 may be configured to engage with a bottom surface or edge of the second pocket 380 of the top wall 300. The hook portion 570 may be configured to retain the releasable tube retaining means 500 in an assembled configuration with the top wall 300. Hook portion 570 may be configured to engage with a lower surface of the second pocket 380 to retain the first and second locking members 540, 560 within the first and second pockets 370, 380 in use. The second segment 574 of the hook portion 570 may be configured to catch on the lower surface or edge of the second pocket 380 and prevent upwards movement of the releasable tube retaining means 500, when the apparatus 600 is assembled.

The tube engagement portion 562 of the second locking member 560 may be provided on a pressure control tube 400 facing side of the leg portion 568. In the illustrated embodiment the tube engagement portion 562 is provided by the lower portion 578 of the leg portion 568 and the first segment 572 of the hook portion 570. The tube engagement portion 562 may comprise a second contact surface 569. The second contact surface 569 may be configured to be a corresponding mating surface with the upper and lower surface 436, 438 of the engagement features 430. The second contact surface 569 may be configured to be selectively engageable with at least one engagement feature 430.

The second contact surface may substantially be a mirror image of the upper and lower surfaces 436, 438 of the engagement features 430. The corner 579 may form part of the second contact surface 569. The corner 579 may have an internal angle substantially the same as or comparable to angle α of the engagement features 430. The second contact surface 569 may be configured to selectively contact, mate and/or engage with the upper surface 436 and lower surface 438 of the engagement features 430.

The first locking member 540 and second locking member 560 or more particularly the tube engagement portions 542, 562 may each be configured to be selectively engageable with at least one of the plurality of engagement features 430. Engagement between the first locking member 540 and/or the second locking member 560 and any of the engagement features 430 may cause resistance to movement of the pressure control tube 400 within the pressure regulator device 200, chamber 210 and/or body of water. The resistance to movement of the pressure control tube 400 may secure, lock, or hold the pressure control tube 400 at a user-defined height, depth, position, or pressure setting within the pressure regulator device 200.

As shown in FIG. 16, the second locking member 560 may be longer than the first locking member 540. As such, the tube engagement portion 562 of the second locking member 560 may be configured to engage with a different engagement feature 430 to the tube engagement portion 542 of the first locking member 540. The tube engagement portion 562 may be configured to engage with an engagement feature 430 that is below another engagement feature 430 that the tube engagement portion 542 is configured to engage with.

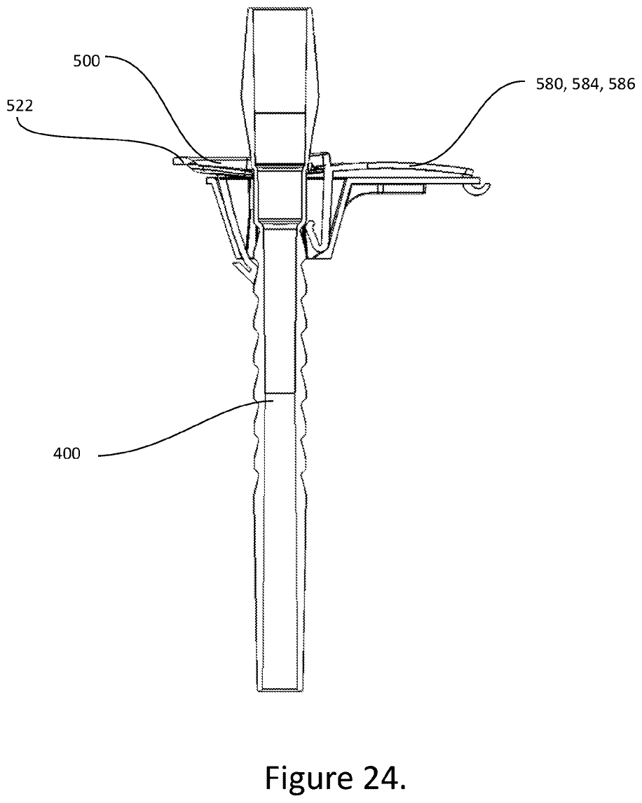

As shown in FIGS. 20 to 21 and FIGS. 24 to 26, the first locking member 540 may be configured to have a length shorter than a height of the first pocket 370. The length of the first locking member 540 may be such that the hook portion 550 of the first locking member 540 is fully received or contained within the first pocket 370, in use. This may allow the first locking member 540 to move or be lifted or lowered within the first pocket when the tube retaining means 500 is adapted between the first and second configurations. The second locking member 560 may be configured to have a length greater than the second pocket 380. The length of the second locking member 560 may be such that the tube engagement portion 562 and hook portion 570 are positioned below or external to the second pocket 580 in use. This may enable the hook portion 570 to flex away from the pressure control tube 400, in use.

In the embodiment shown, the tube engagement portion 562 of the second locking member 560 and the tube engagement portion 542 of the first locking member 540 are spaced apart vertically or in height by a distance that is the same as the spacing between each of the engagement features 430. This results in the tube engagement portion 562 being engageable with an engagement feature 430 that is immediately below (i.e. the next engagement feature 430 down) the engagement feature 430 that the tube engagement portion 542 is engageable with. In alternative embodiments, the first and second locking members 540, 560 may be configured to engage with the same engagement feature 430, or the tube engagement portions 542, 562 may be spaced apart such that they engage with engagement features 430 that are spaced apart by one or more other engagement features 430.

The corners 556 and 579 of the tube engagement portions 542, 562 may be laterally spaced apart by a distance D, such as shown in FIG. 17. The distance D may be substantially the same as a diameter of the pressure control tube 400 at the corner 442 of the engagement features 430. The minimum diameter of the pressure control tube may be defined by the recessed feature 440. The distance D between the corners 556, 579 of the first and second locking members 540, 560 may be greater than the minimum diameter of the pressure control tube 400. The distance D may be less than a maximum diameter of the shaft or body portion 422 of the pressure control tube 400. The maximum diameter of the body portion 422 may be defined by the outer surface 424. A distance D less than the maximum diameter of the body portion 422 may result in the locking members 540, 560 being deformed, deflected, or urged outward by the pressure control tube 400 when they are misaligned with the engagement features 430.

### Operation of Releasable Tube Retaining Means

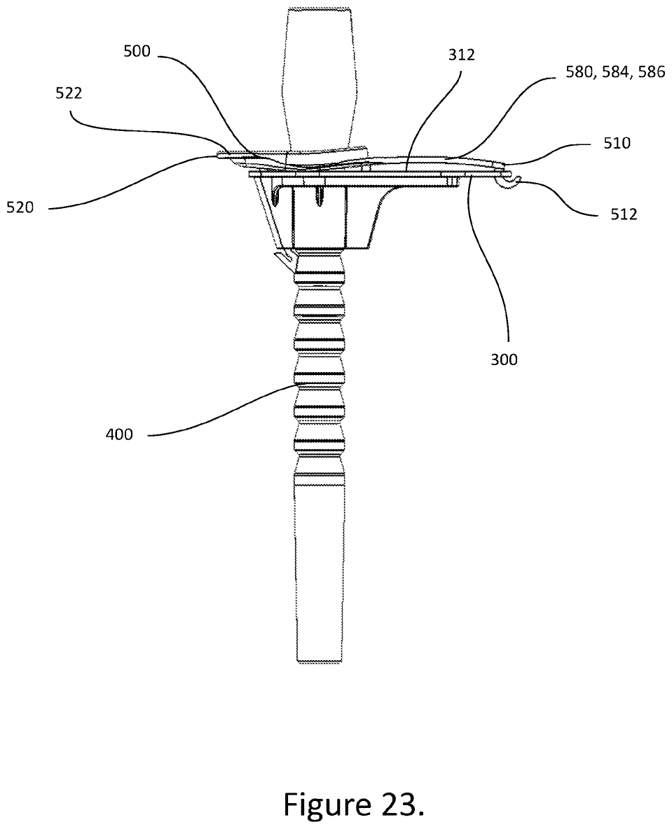

FIGS. 18 to 29 show various views of the apparatus or device 600 of FIGS. 3 and 4 with the top wall 300 shown partially cut-away. It is to be understood that, in reality, the top wall 300 as shown in FIGS. 18 to 21 would extend beyond the boundaries as shown and include other features such as, but not limited to, those described in relation to FIGS. 5 to 9.

The releasable tube retaining means 500 may be configured to be selectively adaptable between a first configuration and a second configuration. When in the first configuration, the releasable tube retaining means 500 may be configured to engage with the pressure control tube 400 and resist movement of the pressure control tube 400 within the pressure regulator device 200. When in the second configuration, the releasable tube retaining means 500 may be deformed such that resistance to movement of the pressure control tube 400 may be reduced relative to the first configuration. This may allow a user to adjust a height or position of the pressure control tube 400 with a reduced force, and thus more easily.

FIGS. 18-21 show the releasable tube retaining means 500 in the first configuration. The first configuration may be a substantially neutral position, state, or configuration, wherein the releasable tube retaining means 500 is not under any external forces applied by a user. When in the first configuration, the releasable tube retaining means 500 may be in a shape or configuration that is substantially the same as when it is moulded or formed.

In the first configuration, the releasable tube retaining means 500 may be engaged with the pressure control tube 400 such that the pressure control tube 400 is locked or retained in a user-defined position within the pressure regulator device 200. When the pressure control tube 400 is locked or retained in a user-defined position, movement of the pressure control tube 400 within the pressure regulator device 200 may be resisted by a resistance force. The resistance to movement of the pressure control tube 400 may be such that the pressure control tube 400 may not be raised or lowered within the chamber 210 or body of liquid unless a user applies a predetermined force that is sufficient to overcome the resistance force.

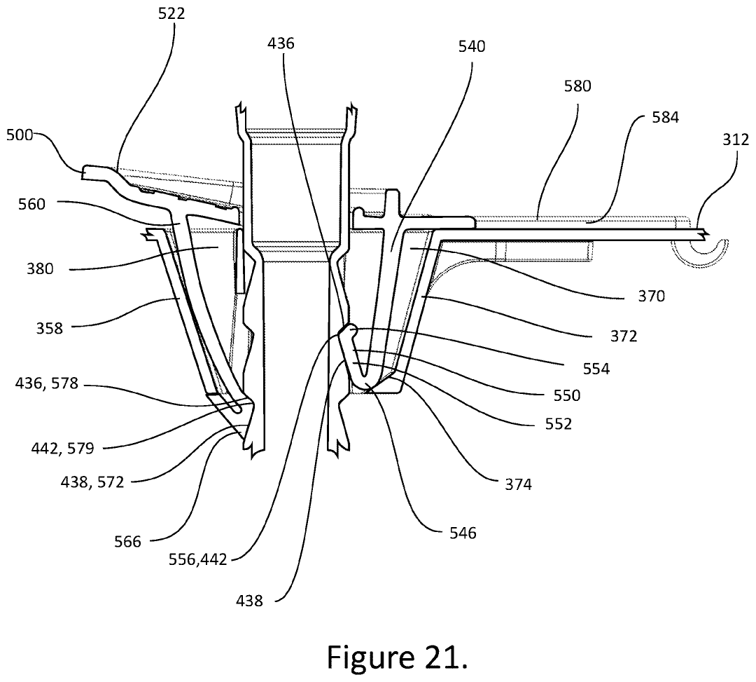

When the releasable tube retaining means 500 is in the first configuration, the first locking member 540 and the second locking member 560 may be in a first position, as shown in FIG. 21. In the first position, the first locking member 540 and the second locking member 560 may be engaged with at least one of the engagement features 430 of the pressure control tube 400. When the first and second locking members 540, 560 are in the first position, their respective tube engagement portions 542, 562 may vertically aligned and/or mated with an engagement feature 430. When the tube engagement portions 542, 562 are in the first position and mated and/or engaged with an engagement feature 430, the first and second contact surfaces 558, 569 may be in contact with the upper and lower surfaces 436, 438 of the engagement feature 430.

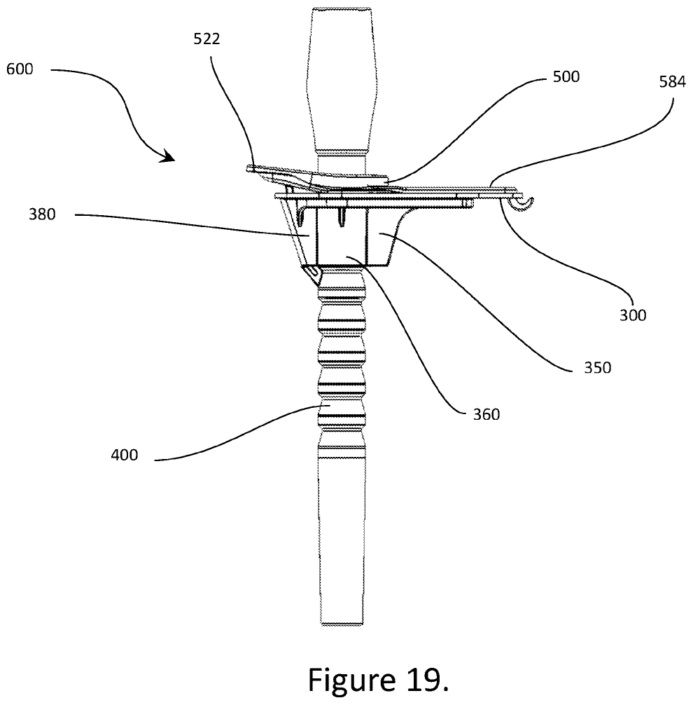

FIGS. 19 and 20 show that when the releasable tube retaining means 500 is in the first configuration the resiliently deformable region 580, including spring arms 584, 586, may be in an un-deformed state or neutral position. When in an un-deformed state or neutral position the resiliently deformable region 580 may be substantially flat and may lie substantially flush with the top surface 312 of the top wall 300.

When the releasable tube retaining means 500 is in the first configuration, the actuating member 522 may be in an un-deformed, undepressed, or neutral position or state. When in an un-deformed, undepressed or neutral position or state, the actuating member 522 may be angled upwardly away from the top surface 312 of the top wall 300. The second end 510 of the elongate body 502 may be spaced apart from the top surface 312 of the top wall 300 when the releasable tube retaining means 500 is in the first configuration.

FIG. 21 shows an embodiment of the releasable tube retaining means 500 in the first configuration. In the first configuration, the tube engagement portion 542 or hook portion 550 of first locking member 540 may be in a first position. In the first position, the tube engagement portion 542 or hook portion 550 may be in substantial alignment and/or mated engagement with one of the engagement features 430. When the hook portion 550 is aligned with an engagement feature 430, the first segment 552, corner 556 and second segment 554 may be vertically aligned or vertically centred with the upper surface 436, corner 442 and lower surfaces 438 the engagement feature 430 respectively. In other words, the engagement portion 542 or hook portion 550 may be at a same or similar height as one of the engagement features 430. Furthermore, the first contact surface 558 of the hook portion 550 may be vertically aligned and mated or in contact or engagement with the upper and lower surfaces 436, 438 of the engagement feature 430 when the releasable tube retaining means 500 is in the first configuration.

When the releasable tube retaining means 500 is in the first configuration, the tube engagement portion 562 of second locking member 560 is in a first position. In the first position, the tube engagement portion 562 may be in substantial alignment and/or mated engagement with one of the engagement features 430. When the tube engagement portion 562 is aligned with an engagement feature 430, the lower portion 578, corner 579 and first segment 572 may be vertically aligned or vertically centred with the upper surface 436, corner 442 and lower surfaces 438 the engagement feature 430 respectively. In other words, the engagement portion 562 may be at a same or similar height as one of the engagement features 430. Furthermore, the second contact surface 569 of the tube engagement portion 562 may be vertically aligned and mated or in contact or engagement with the upper and lower surfaces 436, 438 of the engagement feature 430 when the releasable tube retaining means 500 is in the first configuration.

When the releasable tube retaining means 500 is in the first configuration, engagement between the tube engagement portions 542, 562 of the first and second locking members 540, 560 and the one or more engagement features 430 of the pressure control tube 400 may cause resistance to movement of the pressure control tube 400 within the pressure regulator device 200.

The angle of the first and second locking members 540, 560 and the spacing between the corners 556 and 579 of the tube engagement portions 542, 562, may cause the tube engagement portions 542, 562 to be biased towards and/or bear against the pressure control tube 400 in use.

A contact area may be defined between each of the tube engagement portions 542, 562 and one of the engagement features 430 of the pressure control tube 400 where they contact or engage with each other. A size of the contact area may be determined by an area of the first or second contact surfaces 558, 569 that is in direct contact or engagement with the upper or lower surfaces 436, 438. The contact area may be largest when the releasable tube retaining means is in the first configuration and the tube engagement portions 542, 562 are substantially aligned, mated, or fully engaged. As shown in FIG. 21, the first and second contact surfaces 558, 569 may both be substantially fully engaged or mated with the upper and lower surfaces 436, 438 of an engagement feature 430 when in the first position. The size of the contact area may at least in part determine a resistance force with which the first and/or second locking members 540, 560 resists movement of the pressure control tube 400. A larger contact area may result in a greater resistance to movement of the pressure control tube 400 than a smaller contact area.

The first and second locking members 540, 560 and their associated tube engagement portions 542, 562 may be configured to engage with an engagement feature 430 such that a force applied to the pressure control tube 400 in an upwards or downwards direction is transferred from the engagement feature to the tube engagement portions 542, 562 of the locking members.

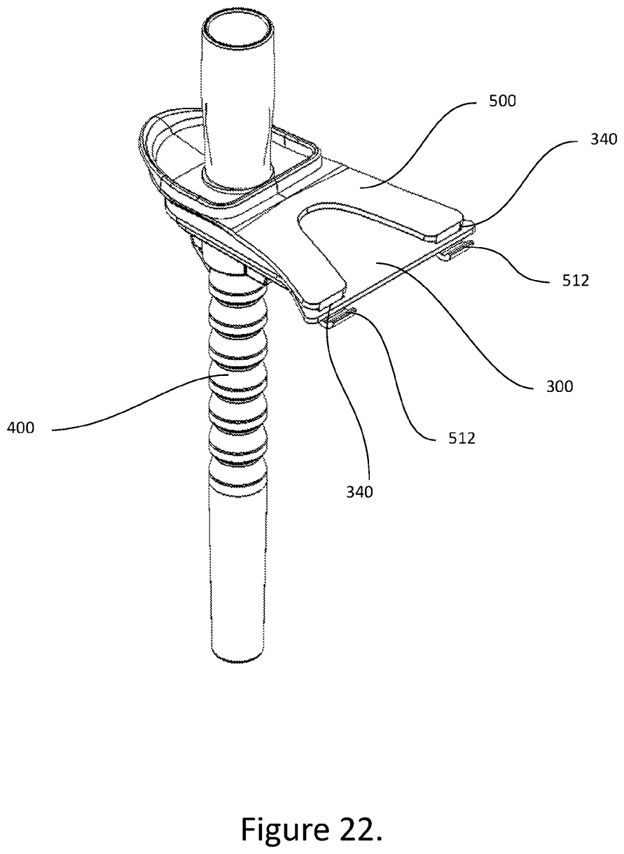

FIGS. 22 to 25 show an embodiment of the releasable tube retaining means 500 in the second configuration. The second configuration may be a deformed, actuated or released position, state, or configuration. The releasable tube retaining means 500 may be adapted to the second configuration when a user applies a downwardly directed actuating force to the actuating member 522. The application of a downwards force to the actuating member 522 may urge or depress the actuating member 522 and the second end 520 towards the top wall 300 and deform the releasable tube retaining means 500.

In the second configuration, engagement between the releasable tube retaining means 500 and the pressure control tube 400 may be reduced or at least partially released such that resistance to movement of the pressure control tube is reduced. Resistance to movement of the pressure control tube 400 may be reduced relative to when the tube retaining means 500 is in the first configuration. The resistance to movement of the pressure control tube 400 may be reduced but not eliminated. The reduced engagement between the releasable tube retaining means 500 and the pressure control tube 400 may make it easier for a user to adjust the position of the pressure control tube 400 within the pressure regulator device 200.

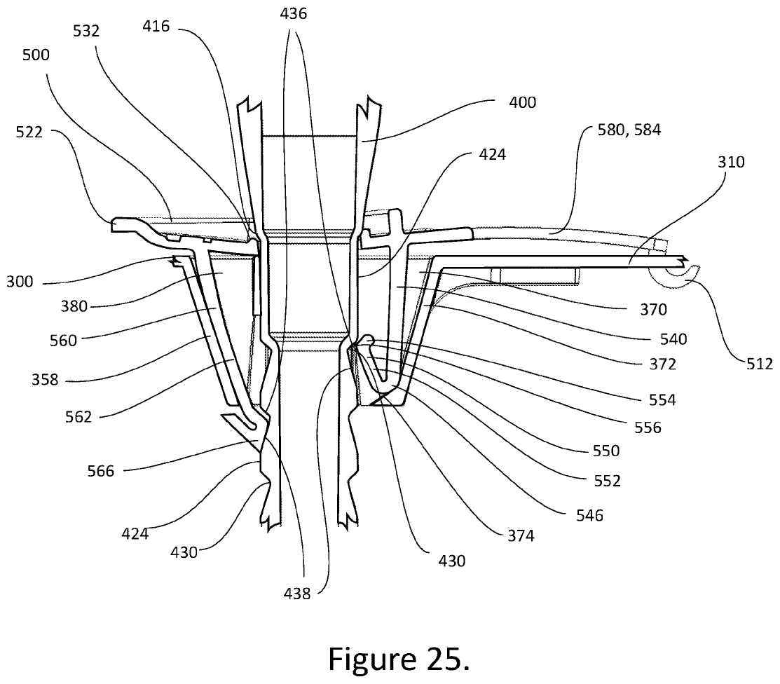

When the releasable tube retaining means 500 is in the second configuration, the first and/or second locking members 540, 560 may be at least partially disengaged from the engagement features 430 of the pressure control tube 400. When the first and/or second locking members 54, 560 are at least partially disengaged from the engagement features 430 the resistance to movement of the pressure control tube may be reduced relative to when the tube retaining means is in the first configuration. As shown in FIG. 25, the tube engagement portions 542, 562 may be moved out of alignment such that they are no longer mated with an engagement feature 430. When the tube engagement portions 542, 562 are misaligned with the engagement features 430, a contact area between the tube engagement portions 542, 562 and the pressure control tube 400 may be reduced. This reduces the resistance to movement of the pressure control tube 400 within the pressure regulator device 200 or body of liquid. In the second configuration, the releasable tube retaining means 500 may still resist movement of the pressure control tube 400, but with a reduced resistance force relative to the first configuration. The resistance to movement of the pressure control tube 400 in the second configuration may be such that the pressure control tube 400 may be raised or lowered within the chamber 210 or body of liquid when a user applies a force that is sufficient to overcome the reduced resistance force. In other words, the force required to move the pressure control tube 400 within the pressure regulator device 200 is reduced when the releasable tube retaining means 500 is adapted to the second configuration. This allows a user to adjust the position of the pressure control tube 400 and thus a pressure of the gases delivered to a patient by the pressure regulator device 200, more easily.

As shown in FIG. 25, when the actuating member 522 is urged or depressed towards the top surface 312 of the top wall 300, the second end 520 of the releasable tube retaining means 500 pivots about the hinge/fulcrum 508. The releasable tube retaining means 500 is constrained at the first end 510 by the coupling portion 312 or hinge hooks 514 being coupled with coupling feature 340 of the top wall 300. This constraint causes the resiliently deformable region 580, including spring arms 584, 586, to deform and/or bow upwards away from the top surface 312, when the actuating member 522 is urged towards the top surface 312. The resiliently deformable region 580 and/or spring arms 584, 586 may arc or bow upwardly between the hinge/fulcrum 508 and the coupling portion 312.

In the second configuration, upwards bowing of the resiliently deformable region 580 may cause the first locking member 540 to be lifted or urged upwardly, towards the top wall 300. As the first locking member 540 is lifted or urged upwards, the distal end 546 may contact and slide up the ramp surface 374 of the first pocket 370. As the distal end 546 slides up the ramp surface 374, the tube engagement portion 542 or hook portion 550 can move out of vertical alignment and laterally away from engagement feature 430. When the tube engagement portion 542 is lifted upwards out of alignment and away from the engagement feature 430 the first contact surface 558 and/or hook portion 550 can be fully disengaged from the engagement feature (as shown in FIG. 25) or alternatively can remain at least partially engaged with the engagement feature 430.

The resiliently deformable region 580 may be configured to provide a re-locking force. The re-locking force may be provided by stored energy within the resiliently deformable region 580 when it is adapted to the second configuration. The re-locking force may urge the resiliently deformable region 580 to return to an un-deformed position or state. The re-locking force may cause the releasable tube retaining means 500 to be biased towards the first configuration.

The force applied to the actuating member 522 in combination with the specific geometry of the releasable tube retaining means 500 may determine whether the tube engagement portion 542 and hook portion 550 remains in at least partial engagement or is completely disengaged from the engagement features 430. When the tube engagement portion 542 and hook portion 550 are completely disengaged from the engagement feature 430, the first locking member 540 may provide little or no resistance to movement of the pressure control tube 400 within the pressure regulator device 200.

In embodiments where the tube engagement portion 542 and hook portion 550 remain in at least partial engagement with the engagement feature 430, the first contact surface 558 may be spaced away from the lower surface 438 while remaining in contact or engagement with at least a portion of the upper surface 436 or the outer surface 424 of the pressure control tube 400. The first segment 552 of the hook portion 550 may be spaced away from the lower surface 438 of the engagement feature 430. The first segment 554 and/or corner 556 may remain in contact or engagement with at least a portion of the upper surface 436 of the engagement feature 430 or the outer surface 424 of the pressure control tube. The engagement between the hook portion 550 and the engagement feature 430 may be located at or near an outer edge of the upper surface 436 adjacent the outer surface 424 or on the outer surface 424. Such partial contact or engagement between the tube engagement portion 542 and an engagement feature 430 may cause minimal resistance to movement of the pressure control tube 400 within the pressure regulator device 200.

As shown in FIG. 25, when the actuating member 522 is urged towards the top wall 300, the second locking member 560 may be urged downwardly and into engagement with the lower surface 438 of one of the engagement features 430. The angle of the lower surface 438 in combination with the angle of the second contact surface 569 of the tube engagement portion 562 may cause the tube engagement portion 562 to slide downwardly along the lower surface 438 and out of vertical alignment with the engagement feature 430. The tube engagement portion 562 of the second locking member 560 may remain in contact and/or engagement with pressure control tube 400 due to an angle of the second locking member 560 relative to the elongate body 502 and the pressure control tube 400.

When the releasable tube retaining means 500 is in the second configuration, the lower portion 578 of the leg portion 568 may be urged downwardly away from and out of contact or engagement with the upper surface 436 of the engagement feature 430. The first segment 572 of the hook portion 570 may remain at least partially in contact or engagement with the lower surface 438 and/or the outer surface 424 of the pressure control tube 400, as shown in FIG. 25.

It may be advantageous for the tube engagement portion 562 of the second locking member 560 to remain at least partially engaged with the engagement feature 430. Partial engagement may provide a level of resistance to movement of the pressure control tube 400 that is sufficient to minimize the risk of the pressure control tube 400 falling or being dropped into the chamber 210 if a user lets go of the pressure control tube 400 when the tube retaining means 500 is in the second configuration. In other words, the second locking member 560 remains in at least partial engagement with an engagement feature 430 and provides resistance to movement of the pressure control tube 400 when the tube retaining means 500 is adapted to the second configuration.

The second end wall 358 of the second pocket 380 of the guide portion 350 may be configured to constrain the movement of the second locking member 560. The second end wall 358 may be configured to constrain or restrict how far the second locking member 560 can deflect away from the pressure control tube 400. The second end wall 358 may be configured to maintain contact and/or engagement between the tube engagement portion 562 and the pressure control tube 400.

In the illustrated embodiment, the leg portion 568 of the second locking member 560 is curved and configured to be positioned adjacent and partially in contact with the second end wall 358 in use. The curved structure of the leg portion 568 in combination with the constraint provided by the second end wall 358 results in the second locking member 560 acting as a spring to bias the tube engagement portion 362 towards the pressure control tube 400. The curved structure of the leg portion 568 also allows the distal end 566 to be deflected or deformed away from the pressure control tube 400 in use. The second end wall 358 may restrict how far the distal end 566 can deflect from the pressure control tube 400.

The second locking member 560 can act as a detent member or ratchet-like arrangement that restricts movement of the pressure control tube 400 within the bubble CPAP pressure regulator device 200. The detent or ratchet arrangement provides a variable resistance to movement of the pressure control tube 400.

### Adjusting Pressure

A pressure setting of the pressure regulator device 200 may be changed by adjusting the position of the pressure control tube 400 within the pressure regulator device 200. Users may set the pressure of gases delivered to a patient by positioning the pressure control tube 400 at a user-defined position within the pressure regulator device 200. The user may position the pressure control tube 400 at one of one or more discrete positions, heights, depths of submersion or settings that corresponds to a desired pressure. The position of the pressure control tube 400 may be adjusted by applying an upwards or downwards force to the pressure control tube 400. The position of the pressure control tube 400 may be adjusted when the releasable tube retaining means 500 is in either the first configuration or the second configuration. The force required to adjust the position of the pressure control tube 400 when the tube retaining means 500 is in the first configuration may be greater than when the tube retaining means 500 is in the second configuration. Thus, it may be easier to adjust the position of the pressure control tube 400 when the tube retaining means 500 is in the second configuration.

A user can adjust the position of the pressure control tube 400 within the pressure regulator device 200 by adapting the releasable tube retaining means 500 to the second configuration and applying a force to the pressure control tube 400. The tube retaining means 500 may be adapted to the second configuration by a user applying an actuating force to the actuating member 522 that urges the actuating member 522 towards the top wall 300. The pressure control tube 400 may then be moved by the user gripping the grip portion 412 and applying an upwards or downwards force.

A user can also adjust the position of the pressure control tube 400 when the tube retaining means 500 is in the first configuration. The pressure control tube may be moved by the user gripping the grip portion and applying an upwards or downwards force. A greater force may be required to adjust the position of the pressure control tube 400 when the tube retaining means 500 is in the first configuration than when the tube retaining means 500 is in the second configuration.

The releasable tube retaining means 500 may be configured to resist movement of the pressure control tube 400 until a force is applied to the pressure control tube 400 that is greater than a resistance force provided by the first and second locking members 540, 560. The resistance force provided by the first and second locking members 540, 560 may be a function of one or more of the angles, thickness and/or material of the first and second locking members 540, 560, tube engagement portions 542, 562, the ramp surface 374, and/or the angles of the upper and lower surfaces 436, 438 of the engagement features 430 of the pressure control tube 400.

With the releasable tube retaining means 500 in the first configuration, the position of the pressure control tube 400 may adjusted by applying a first downstroke or upstroke force to the pressure control tube 400. The first downstroke force may be applied by pushing or urging the pressure control tube downwards into the chamber 210 and body of liquid. The first upstroke force may be applied by pulling or lifting the pressure control tube upwards out of the chamber 210 and body of liquid. The first downstroke and first upstroke forces may be sufficient to overcome the resistance to movement provided by engagement of the first and second locking members 540, 560 with the engagement features 430 of the pressure control tube 400.

When the tube retaining means 500 is in the first configuration, the tube engagement portions 542, 562 of the first and second locking members 540, 560 are each vertically aligned and in mated engagement with an engagement feature 430 of the pressure control tube 400. The application of the first downstroke force to the pressure control tube 400, may cause the engagement feature 430 to apply a downwardly directed compressive force to the tube engagement portions 542, 562. In the illustrated embodiment the upper surfaces 436 of the engagement features 430 apply a compressive force to the second segment 554 of the hook portion 550 of the first locking member 540 and to the lower portion 578 of the leg portion 568 of the second locking member 560. The tube engagement portions 542, 562 resist the compressive force until the compressive force is great enough to deform or deflect the tube engagement portions 542, 562 and/or the distal ends 546, 566 of the first and second locking members 540, 560 outwardly and away from the pressure control tube 400.

The angled surfaces of the upper surface 436 and the first and second contact surfaces 558, 569 may act as ramps or inclined planes. The ramps or inclined planes may cause the tube engagement portions 542, 562, hook portion 550 and/or distal ends 546, 566 of locking members 540, 560 to slide or deflect away from the pressure control tube 400. Movement of the pressure control tube 400 is resisted until the compressive force reaches or exceeds the first downstroke force. When the tube engagement features 542, 562 are deflected outwardly, their engagement with the pressure control tube 400 reduced and thus the resistance to movement of the pressure control tube 400 is reduced and the pressure control tube 400 can move downwards.

The application of the first upstroke force to the pressure control tube 400 may cause the engagement feature 430 to apply an upwardly directed compressive force to the tube engagement portions 542, 562. In the illustrated embodiment, the lower surfaces 438 of the engagement features 430 apply a compressive force to the first segment 552 of the hook portion 550 of the first locking member 540 and to the first segment 572 of the hook portion 570 of the second locking member 560. The tube engagement portions 542, 562 may resist the compressive force until the compressive force is great enough to deform or deflect the tube engagement portions 542, 562 and/or the distal ends 546, 566 of the first and second locking members 540, 560 outwardly and away from the pressure control tube 400. Movement of the pressure control tube 400 is resisted until the compressive force reaches or exceeds the first upstroke force. When the tube engagement features 542, 562 are deflected outwardly, their engagement with the pressure control tube 400 reduced and thus the resistance to movement of the pressure control tube 400 is reduced and the pressure control tube 400 can move upwards.

When the releasable tube retaining means 500 is in the second configuration, the position of the pressure control tube 400 may adjusted by applying a second downstroke force or a second upstroke force to the pressure control tube 400. The second downstroke force may be applied by pushing or urging the pressure control tube downwards into the chamber 210 and body of liquid. The second upstroke force may be applied by pulling or lifting the pressure control tube upwards out of the chamber 210 and body of liquid. The second downstroke and second upstroke forces may be sufficient to overcome the reduced resistance to movement provided by the first and second locking members 540, 560 when they are at least partially disengaged from the engagement features 430 of the pressure control tube 400.

When the tube retaining means 500 is in the second configuration the tube engagement portion 542 of the first locking member 540 may be partially or completely disengaged from the pressure control tube 400. The tube engagement portion 562 of the second locking member 560 may be misaligned and at least partially disengaged with the engagement features 430 of the pressure control tube 400. In other words, the tube engagement portions 542, 562 are urged outwardly away from the pressure control tube 400 but may remain at least partially in contact or engaged with the pressure control tube 400 when the releasable tube retaining means 500 is in the second configuration.

As shown in FIG. 25, the first locking member 540 may be completely disengaged from the engagement features 430 such that the first locking member 540 provides little or no resistance to movement of the pressure control tube, when the tube retaining means 500 is in the second configuration. In alternative embodiments or if the actuating member 522 is not fully depressed to the second configuration, the tube engagement portion 542 of the first locking member 540 may remain in partial contact or engagement with the pressure control tube 400. In such an embodiment or arrangement, the tube engagement portion 542 may provide a resistance to movement of the pressure control tube 400 that is reduced relative to when the tube retaining means 500 is in the first configuration. In the illustrated embodiment the tube engagement portion 562 of the second locking member 560 is misaligned with the engagement feature 430 so that the lower end portion 578 of the leg portion 568 of the second locking member 560 is disengaged from the upper surface 436 of the engagement feature 430. This may reduce the resistance to movement of the pressure control tube 400.

Application of the second downstroke force to the pressure control tube 400, when the tube retaining member 500 is in the second configuration, may cause the engagement feature 430 to apply a downwardly directed compressive force to the tube engagement portions 542, 562. The upper surface 436 of the engagement feature 430 may not apply any force to the tube engagement portion 542 if the tube engagement portion 542 is fully disengaged from the pressure control tube 400. If the tube engagement portion 542 is partially in contact or engagement with the pressure control tube 400 then the upper surface 436 may apply a compressive force to the second segment 554, or the outer surface 424 may apply a lateral force to the corner 556 of the tube engagement portion 542. The tube engagement portion 542 and first locking member may provide minimal resistance to movement of the pressure control tube 400.

When the tube retaining means 500 is in the second configuration, the tube engagement portion 562 and the second locking member 560 may provide most of the resistance to movement of the pressure control tube. When the tube retaining means is initially adapted to the second configuration, the lower portion 578 of the leg portion 568 of the second locking member 560 may be disengaged from the upper surface 436 of the engagement feature 430, as shown in FIG. 25. Thus, resistance to movement of the pressure control tube 400 may at least initially be provided by the first segment 572 of the hook portion 570 being urged into engagement with the lower surface 438 of an engagement feature 430 by the depression of the actuating member 522. Thus, initially the tube engagement portion 562 may apply a compressive force to the lower surface 438.

As the second downstroke force is applied to the pressure control tube 400, the compressive force applied to the lower surface 438 by the tube engagement portion 562 is overcome and the pressure control tube 400 can move downwardly. The angle of the lower surface 436 and the surface of the lower portion 578 of the hook portion 570 may cause the compressive force therebetween to increase, deflecting the tube engagement portion 562 of the second locking member 560 out of engagement with the engagement feature 430, until the pressure control tube 400 moves downwardly and an upper surface 436 of an engagement feature 430 comes into engagement with the lower portion 578. Once the pressure tube 400 has been moved downwardly and an upper surface 436 has been urged into engagement with the lower portion 578, a compressive force may be applied by the upper surface 436 to the lower portion 578. Movement of the pressure control tube 400 may once again be resisted until the compressive force equals or overcomes the second downstroke force. When the compressive force reaches or exceeds the second downstroke force, the tube engagement portion 562 and second locking member 560 may be deformed or deflected outwardly away from the pressure control tube 400. When the tube engagement feature 562 is deflected outwardly, engagement with the pressure control tube 400 is reduced and thus the resistance to movement of the pressure control tube 400 is reduced and the pressure control tube 400 can move downwards.

When the second upstroke force is applied to the pressure control tube 400 and the tube retaining means 500 is in the second configuration the first locking member 540 may provide minimal or no resistance to movement of the pressure control tube. If the tube engagement portion 542 of the first locking member 540 is fully disengaged from the pressure control tube 400, as shown in FIG. 25, the first locking member may provide no resistance to upward movement of the pressure control tube. If the tube engagement portion 542 of the first locking member 540 is partially disengaged from the pressure control tube 400, the first locking member 540 may provide minimal resistance to upward movement of the pressure control tube 400. The pressure control tube 400 may be urged upwardly such that the lower surface 438 of an engagement feature 430 applies a generally upwardly directed compressive force to the first segment 572 of the hook portion 570 of the second locking member 560. The second upstroke force may be in a substantially vertical or upwards direction that may be parallel to a central axis of the pressure control tube 400.

The second upstroke force may generate a resultant force vector. The resultant force vector may be a function of the size of the second upstroke force, the coefficient of friction of the surfaces of the pressure control tube 400 and tube engagement portion 542, and the angle θ of the lower surface 438 and the portion of the contact surface 569 that is provided on the first segment 572. The resultant force vector may have a vertical component that is in an opposite direction to the second upstroke force. The second upstroke force may increase the compressive force already in place between the first segment 572 and the lower surface 438. Upwards movement of the pressure control tube 400 may be resisted while the vertical component of the resultant force vector equals or exceeds the second upstroke force. When the second upstroke force reaches or exceeds the vertical component of compressive force, the tube engagement portion 562 and second locking member 560 may be deformed or deflected outwardly away from the pressure control tube 400. When the tube engagement feature 562 is deflected outwardly engagement with the pressure control tube 400 is reduced and thus the resistance to movement of the pressure control tube 400 is reduced and the pressure control tube 400 can move upwards.

The angles of the upper and lower surfaces 436, 438 of the engagement features 430 may at least partially determine the first and second downstroke and the first and second upstroke forces. In the illustrated embodiment the upper surfaces 436 may have a shallower angle than the lower surfaces 438. Conversely, the lower surfaces 438 may have a steeper angle than the upper surfaces 436. In other words, in the illustrated embodiment, the upper surface 436 has a greater angle relative to the outer surface 424, a central axis of the pressure control tube 400, or vertical than the lower surface 438, i.e. angle β is greater than angle θ, as shown in FIG. 13. The shallower angle of the upper surface 436 may cause greater resistance to downwards movement of the pressure control tube 400 than the resistance to upwards movement provided by the steeper angle of the lower surface 438. Thus, the first downstroke force may be greater than the first upstroke force and the second downstroke force may be greater than the second upstroke force. It may be easier to raise or move the pressure control tube 400 upwards than it is to lower or move the pressure control tube 400 downwards.

The second downstroke force or the second upstroke force required to move the pressure control tube 400 within the pressure regulator device 200 may be such that unintended movements of the pressure control tube 400 may not occur as a result of forces applied by the exhalatory conduit 130 or other system component.

The resilience of the first and second locking members 540 and 560 and their angles relative to the elongate body 502 may cause them to be biased inwardly towards the pressure control tube. The inward biasing of the first and second locking members 540, 560 may cause the tube engagement portions 542, 562 to move in and out of mated engagement with one or more of the engagement features 430 as the pressure control tube is moved upwards or downwards. When the tube retaining means 500 is in the first configuration, the tube engagement portions 542, 562 of both the first and second locking members 540, 560 may move in and out of mated engagement with the engagement features 430. When the tube retaining means 500 is in the second configuration, the tube engagement portion 542 of the first member 540 may remain disengaged from the engagement features 430 during movement of the pressure control tube 400. The tube engagement portion 562 of the second locking member 560 may move in and out of mated engagement with the engagement features 430 as the pressure control tube 400 is moved.

The first locking member 540 may be configured to act as a lock that resists movement of the pressure control tube 400 when in a locked position and allows movement when in an unlocked position. The first locking member 540 may be in the locked position when the tube retaining means 500 is in the first configuration. The first locking member 540 may be in the unlocked position when the tube retaining means 500 is in the second configuration.

The second locking member 560 may be configured to act as a detent feature or member that resists upwards and/or downwards movement of the pressure control tube 400. The second locking member 560 may be configured to act as a detent feature or member when the tube retaining means is in the first or second configuration. The level of resistance to movement that the second locking member 560 can provide may vary depending on whether the tube retaining means 500 is in the first or second configuration. The second locking member 560 may provide a higher level of resistance to movement when the tube retaining means 500 is in the first configuration than when the tube retaining means 500 is in the second configuration.

The apparatus 600 may be configured to allow the position of the pressure control tube 400 to be adjusted by a user using a single hand or two hands. When using only one hand the user may apply the actuating force to the actuating member 522 with their thumb. They may then use at least one finger to grip the pressure control tube 400 and apply an upwards or downwards force. When using two hands, the user may use the thumb or a finger of one hand to apply the actuating force to the actuating member 522. They may then use the other hand to grip the pressure control tube 400 an apply an upwards or downwards force.

The actuation force of the tube retaining means 500 and the forces required to overcome the resistance to movement of the pressure control tube 400 may be at least partially determined by the cross-sectional thicknesses and or lengths of the elongate body 502 and first and second locking members 540, 560. In some embodiments the actuation force required to urge the actuating member 522 towards the top wall 300 and adapt the releasable tube retaining means 500 from the first configuration to the second configuration may be between 5 Newtons (N) to 15N. The actuation force may be at least partially determined by the cross-sectional thickness and/or length of the resiliently deformable region 580 and/or the spring members 584, 586.

The actuating member 522 may be configured to act as a lever. Thus, the actuation force may also be at least partially determined by a length of the actuating member 522. The length of the actuating member may be defined between the hinge/fulcrum 508 and the second end 520.

To fully adapt the tube retaining means 500 to the second configuration, the actuating member 522 may be urged downwards by the actuating force until it is obstructed by the top wall 300. The distance that the actuating member 522 travels towards the top wall 300 may define an actuation stroke length. In some embodiments the actuation stroke length may be between approximately 2 mm and 5 mm.

The first and second downstroke forces and first and second upstroke forces may be determined by several factors. Some of the factors may include the cross-sectional thickness and/or length of the first and second locking members 540, 560, angles of the upper and lower surfaces 436, 438 of the engagement features 430, angles of the first and second contact surfaces 558, 569 and material of the tube retaining means 500. Other factors may also be varied to determine the downstroke and upstroke forces. Such factors may include the coefficient of friction of the surfaces of the pressure control tube 400 and tube engagement portions 542, 562, a flexural preload of the first and second locking members 540, 560, and angles and/or positions of the internal surfaces of the first and second end walls 356, 358 including ramp 374.

Moving the pressure control tube 400 upwards may be considered less risky than moving the pressure control tube 400 downwards. This may be because raising the pressure control tube 400 may decrease the pressure of gases delivered to a patient. Decreasing the pressure of gases delivered to a patient is unlikely to cause harm to the patient if it occurs unintentionally and is therefore low risk. Conversely, lowering the pressure control tube may increase the pressure of gases delivered to a patient. Increasing the pressure of gases delivered to a patient may cause damage to a patient's lungs if it were to occur unintentionally and is therefore high risk. It may be advantageous for the upstroke and downstroke forces to be as low as possible to make it easy for a user to adjust the position of the pressure control tube 400. Due to the risks associated with downwards movement of the pressure control tube 400, the downstroke forces may need to be sufficient to resist unintentional downward movement of the pressure control tube 400. In the illustrated embodiment the apparatus 600 is configured such that the first and second upstroke forces are lower than the first and second downstroke forces respectively. In alternative embodiments the first upstroke force may be the same as the first downstroke force and second upstroke force may be the same or similar to the second downstroke force.

In some embodiments the first downstroke force required to overcome the resistance to movement of the pressure control tube 400, when the tube retaining means 500 is in the first configuration, may be greater than approximately 20N. The second downstroke force required to overcome the resistance to movement of the pressure control tube 400, when the tube retaining means 500 is in the second configuration, may be between approximately 10N and 20N.

In some embodiments the first upstroke force may be less than the first downstroke force. In some embodiments the first upstroke force may be greater than approximately 10N. The second upstroke force may be less than the second downstroke force. In some embodiments the second upstroke force may be between approximately 0N and 10N.

### Unstressed State

It may be desirable for the pressure regulator device 200 to be in a fully assembled state when it is packaged for transport and/or storage. This enables a user to set up the pressure regulator 200 for use quickly and easily upon removal from the packaging. It may also be desirable for the pressure regulator device 200 to be as compact as possible, to reduce the size and cost of packaging. The pressure regulator device 200 may be configured to be assembled with the pressure control tube 400 in a fully lowered position when packaged for storage and/or transport.

It may be desirable for the releasable tube retaining means 500 and or pressure control tube 400 to be in a substantially unstressed state when they are assembled and packaged for storage or transportation. This may reduce the risk of creep deformation of the releasable tube retaining means 500 and pressure control tube 400 if the pressure regulator device 200 is in storage or transportation for an extended period. Reducing the risk of creep deformation may also minimise the risk of the pressure control tube 400 dropping in height unintentionally, when in use. The tube retaining means 500 may be in a substantially unstressed state when it is under minimal or no mechanical stress. In the substantially unstressed state, any external forces or loads applied to the tube retaining means 500 and/or pressure control tube 400 may be minimised. The apparatus 600 may be configured to be selectively adaptable to a storage and transport configuration wherein at least the tube retaining means 500 is in a substantially unstressed state.

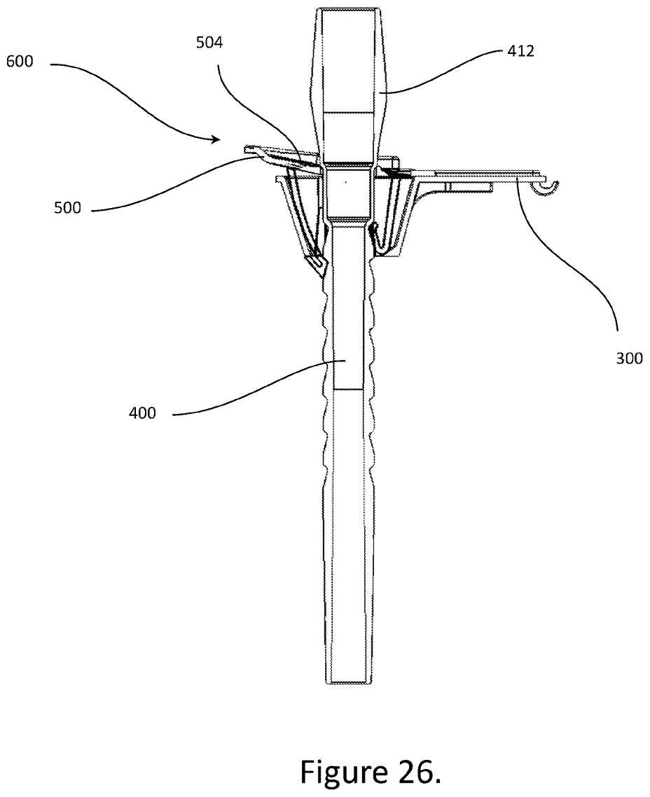

FIG. 26 shows an embodiment of apparatus 600 assembled for storage and transport. In this assembled configuration, the releasable tube retaining means 500 may be coupled to the top wall 300. The pressure control tube 400 may be inserted through the tube receiving portion 530 of the tube retaining means 500 and the tube aperture 360 of the top wall 300. The pressure control tube 400 may be in a fully lowered position. The pressure control tube 400 may be in a fully lowered position when a lower surface of the grip portion 412 is abutted with the bead 532 or upper surface 504 of the tube retaining means 500. The fully lowered position of the pressure control tube 400 may correspond to a maximum depth of submersion in the body of liquid and a maximum pressure setting of the pressure regulator device 200.

The releasable tube retaining means 500 may be adaptable to a substantially unstressed state. In the substantially unstressed state, little or no external forces are applied to the releasable tube retaining means 500 by a user. The locking members 540, 560 are substantially un-deformed by engagement between the free ends and the pressure control tube 400. The releasable tube retaining means 500 may be selectively engageable with the pressure control tube 400 in at least one position in which the releasable tube retaining means 500 is in a substantially unstressed state. The tube retaining means 500 may be adapted to the substantially unstressed state when the apparatus 600 is in the assembled storage and transport configuration.

When in the storage and transportation configuration the releasable tube retaining means 500 may substantially be in the first configuration. The actuating member 522 and resiliently deformable region 580 of the tube retaining means 500 may be in a substantially un-deformed position or state. The releasable tube retaining means 550 may be engageable with the pressure control tube 400 in a substantially unstressed state when the pressure control tube 400 is in the fully lowered position.

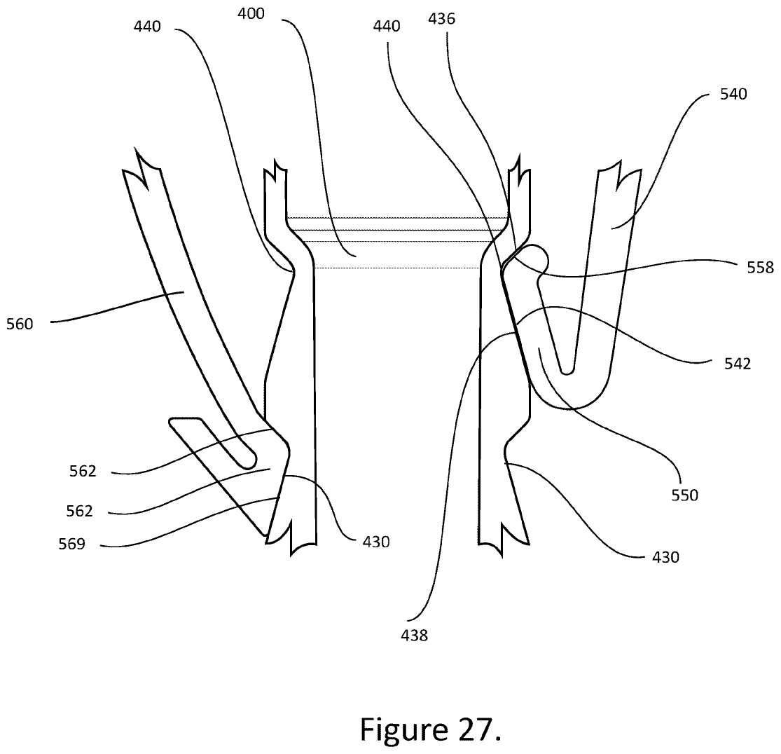

When the apparatus 600 is in the assembled storage and transport configuration, at least a portion of the first locking member 540 may be received in the recessed feature 440 of the pressure control tube 400. The portion of the first locking member 540 received in the recessed feature 440 may comprise the tube engagement portion 542. As shown in FIG. 26, the tube engagement portion 542 of the first locking member 540 is configured to be vertically aligned with the recessed feature 440 when the apparatus 600 is in the storage and transport configuration. The tube engagement portion 542 or hook portion 550 may be at least partially received within the recessed feature 440, as shown in FIG. 27.

The increased depth d2 of the recessed feature 440 (as described above) may enable the tube engagement portion 542 and first contact surface 558 to be at least partially spaced away from a surface of the recessed feature 440, as shown in FIG. 27. The increased depth d2 of the recessed feature 440 results in a surface of the recessed feature 440 being at least partially offset or spaced apart from the first contact surface 558 and tube engagement portion 542. This results in reduced contact or engagement between the tube engagement portion 542 of the first locking member 540 and the pressure control tube 400 when the apparatus 600 is in the storage and transport configuration. This in turn may minimize stress to the first locking member 540.

When the apparatus 600 is in the storage and transport configuration, the second locking member 560 may be at least partially received in or engaged with an engagement feature 430 of the pressure control tube 400. The tube engagement portion 562 of the second locking member 560 may be at least partially received within or engaged with an engagement feature 430. In the illustrated embodiment, the tube engagement portion 562 is configured to be at least partially received within or engaged with an engagement feature located immediately below the recessed feature 440. As shown in FIGS. 26 and 27, the tube engagement portion 562 and second contact surface 569 of the second locking member 560 are configured to be vertically aligned and mated with the engagement feature 430 when the apparatus 600 is in the storage and transport configuration.

As described in relation to FIG. 17, the first and second locking members 540, 560 may be configured such that the tube engagement portions 542 and 562 are spaced apart by a lateral distance D. The distance D is substantially the same as the minimum diameter of the engagement features 430 of the pressure control tube 400. Since the recessed feature 440 has a minimum diameter less than the rest of the engagement features 430, there may be clearance space between the tube engagement portions 542 and 562 when the tube retaining means 500 is in the substantially unstressed state. This means that although the tube engagement portion 562 of the second locking member 560 is configured to be mated and engaged with an engagement feature 430 the pressure control tube 400 is exerting minimal or no force on the tube engagement portion 562 as the tube engagement portion 542 of the second locking member may be at least partially spaced away or disengaged from the pressure control tube 400, when in the substantially unstressed state.

### Apparatus Assembly

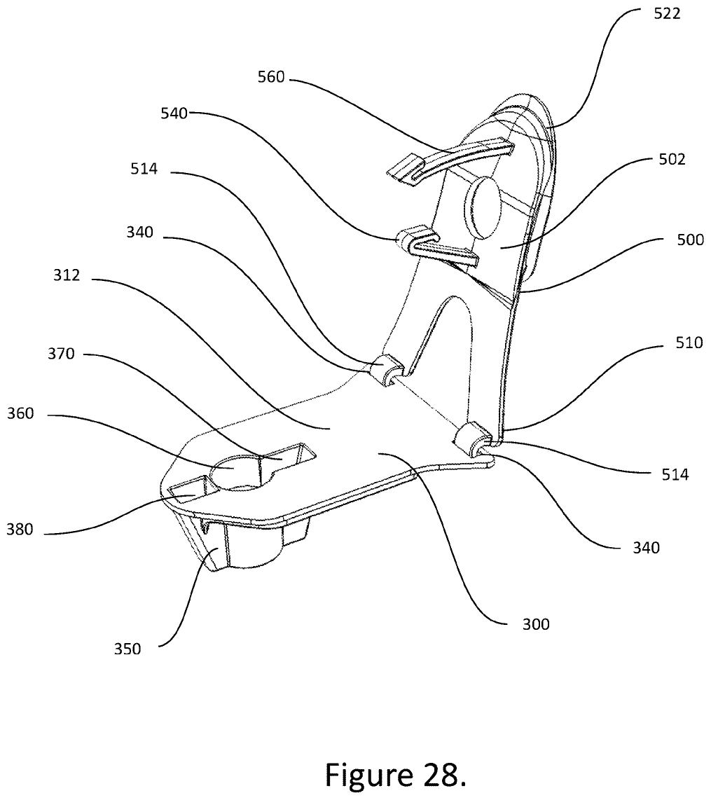

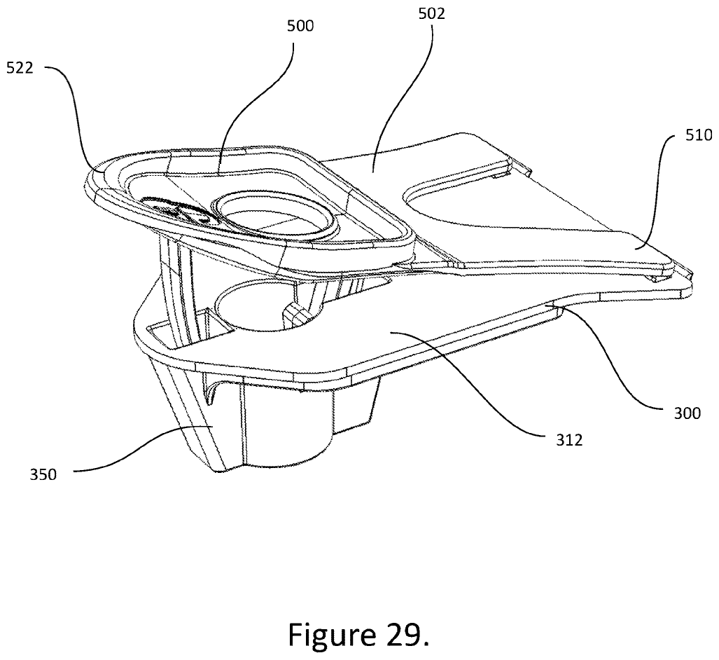

FIGS. 28 to 30 show how the releasable tube retaining means 500 can be assembled to the top wall 300. The coupled end 510 of the tube retaining means 500 may be coupled to the coupling feature 340 of the top wall 300 while the releasable tube retaining means 500 is in an upright or pivoted position, as shown in FIG. 28. The elongate body 502 may be pivoted beyond a vertical position and held at an angle to the top surface 312 so that the terminal end 516 of each of the hinge hooks 514 may be inserted into the slots 342 of the coupling features 340. The tube retaining means 500 can then be pivoted about the coupled end 510 towards the top surface 312, shown in FIG. 29.

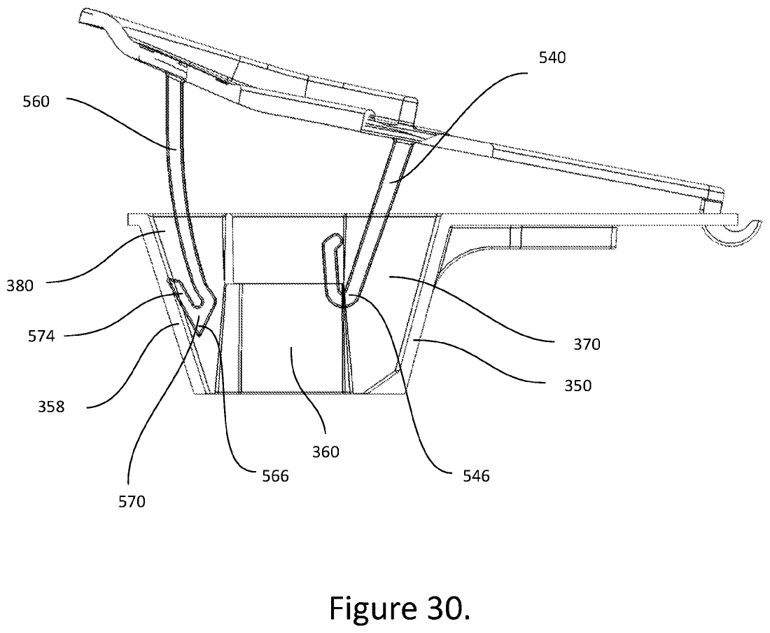

The first locking member 540 and second locking members 560 may be aligned with and inserted into the guide portion 350, as the elongate body 502 of the tube retaining means 500 is pivoted towards the top wall 300. The distal end 546 and tube engagement portion 542 of the first locking member 540 may be pivotally inserted into the first pocket 370. The distal end 566 and the tube engagement portion 562 of the second locking member 560 may be pivotally inserted into the second pocket 380. As shown in FIG. 30, there may be interference between the second segment 574 of the hook portion 570 and the end wall 358 of the second pocket 380 when the tube retaining means 500 is pivoted into engagement with the top wall 300. The second locking member 560 or a portion thereof may be deflected towards the tube aperture 360 by the interference with the end wall 358. The second segment 574 of the hook portion 570 may contact and slide along the end wall 358 as the second locking member 560 enters the second pocket 380.

Once the elongate body 502 has been pivoted into contact with the top surface 312, the distal end 566 and hook portion 550 of the second locking member 560 may protrude through the open bottom of the guide portion 350. When the hook portion 550 is protruding through the open bottom of the guide portion 350, the interference between the second segment 574 and the end wall 358 may be released. The second locking leg 560 may return to its un-deformed state. When the second locking member 560 has returned to its un-deformed state, the second segment 574 may be engaged with a bottom edge or surface of the guide portion 350 and configured to act as a catch that retains the releasable tube retaining means 500 in the assembled position, as shown in at least FIGS. 19 to 21. Once the tube retaining means 500 is assembled with the top wall 300, the pressure control tube 400 may be inserted through the tube receiving portion 530 and tube aperture 360.

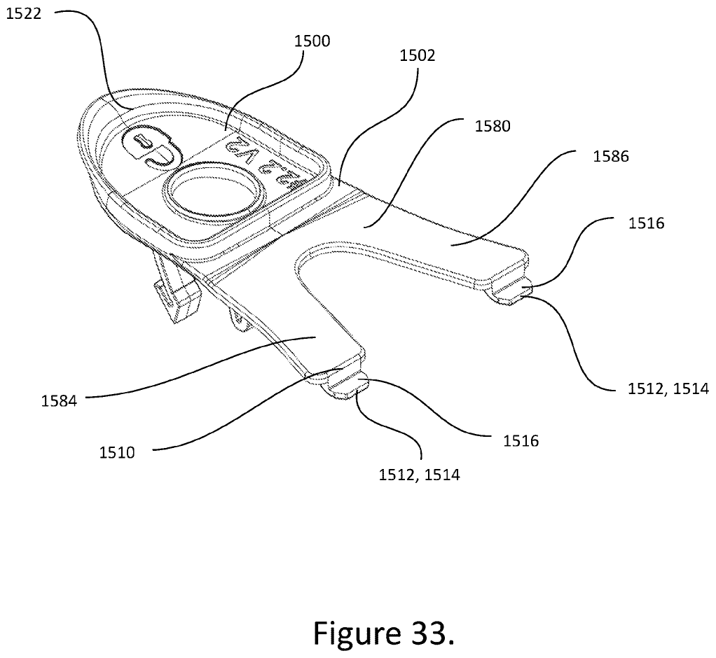

In an alternative embodiment, such as shown in FIGS. 31 to 33, the tube retaining means 1500 may be coupled to the top wall 300 of the pressure regulator system 600 in a similar manner to the tube retaining means 500. The elongate body 1502 may initially be pivoted to or otherwise positioned in a substantially vertical position and held substantially perpendicular to the top surface 312. The second portion of each of the tabs 1514 may then be inserted into the slots 342 of the coupling features 340. The tube retaining means 1500 can then be pivoted about the coupled end 1510 towards the top surface 312, as shown in FIG. 33.

The releasable tube retaining means 500, 1500 may be removed or disassembled from the pressure regulator device 200. To disassemble the tube retaining means 500, 1500 from the top wall 300, the pressure control tube 400 can first be removed or disassembled from the tube receiving portion 530 and tube aperture 360. The catch provided by the second segment 574 of the hook portion 570 may be released by pushing the second segment 574 inwardly towards the tube receiving portion 530. This disengages the second segment from the lower edge or surface of the second pocket and enables the push tab 522 to be lifted and the second locking member 560 lifted out of the second pocket 380. The tube retaining means 500, 1500 can then be pivoted about the first end 510 until the coupling portions 512, 1512 can be removed from the coupling feature 340.

### ALTERNATIVE EMBODIMENT

Although the illustrated embodiment of the releasable tube retaining means 500 comprises first and second locking members 540, 560, in alternative embodiments the releasable tube retaining means 500 may comprise a single, or two or more locking member. In embodiments with a single locking member, the single locking member may perform in substantially the same way as the first locking member 540 as described above. When a tube retaining member 500, having a single locking member 540 is adapted to the second configuration, a tube engagement portion 542 of the single locking member 540 may be configured to remain in partial engagement with the pressure control tube 400. The single locking member 540 may remain in engagement with the pressure control tube 400 to minimise risk of the pressure control tube 400 unintentionally being dropped into the chamber 210.

Where reference is used herein to directional terms such as ‘up’, ‘down’, ‘forward’, ‘rearward’, ‘horizontal’, ‘vertical’ etc, those terms refer to the position and orientation of the apparatus shown in the figures, and are used to show and/or describe relative directions or orientations. Those positions and orientations may be different when the apparatus is in-use.

Although the invention has been described by way of example and with reference to possible embodiments thereof, it is to be understood that modifications or improvements may be made thereto without departing from the scope of the invention. The invention may also be said broadly to consist in the parts, elements and features referred to or indicated in the specification of the application, individually or collectively, in any or all combinations of two or more of said parts, elements or features. Furthermore, where reference has been made to specific components or integers of the invention having known equivalents, then such equivalents are herein incorporated as if individually set forth.

Unless the context clearly requires otherwise, throughout the description, the words “comprise”, “comprising”, and the like, are to be construed in an inclusive sense that is to say, in the sense of “including, but not limited to”, as opposed to an exclusive or exhaustive sense.

Any discussion of the prior art throughout the specification should in no way be considered as an admission that such prior art is widely known or forms part of common general knowledge in the field.

1. An apparatus for retaining a pressure control tube having one or more engagement features in a user-defined position within a pressure regulator device, the apparatus comprising:
a releasable tube retaining means, comprising a resiliently deformable locking member configured to be releasably engageable with at least one of the engagement features such that engagement between the locking member and engagement feature causes resistance to movement of the pressure control tube within the pressure regulator device;
wherein
the releasable tube retaining means is adapted to be selectively engageable with the pressure control tube in at least one position in which the releasable tube retaining means is in a substantially unstressed state.

2. The apparatus of claim 1, wherein at least a portion of the locking member is received in a recessed feature of the pressure control tube when the releasable tube retaining means is in the substantially unstressed state.

3. The apparatus of claim 2, wherein the portion of the locking member that is received in the recessed feature comprises a tube engagement portion.

4. The apparatus of claim 3, wherein the tube engagement portion of the locking member is configured to be at least partially spaced away from a surface of the recessed feature.

5. The apparatus of claim 3, wherein the tube engagement portion comprises a hook portion that extends upwardly towards the pressure control tube in use.

6. The apparatus of claim 3, wherein the tube engagement portion comprises a contact surface configured to mate with a surface of the one or more engagement features of the pressure control tube.

7. The apparatus of claim 6, wherein the contact surface is configured to be at least partially spaced away from a surface of the recessed feature when the releasable tube retaining means is in the substantially un stressed state.

8. The apparatus of claim 2, wherein the recessed feature comprises an engagement feature of the pressure control tube.

9. The apparatus of claim 8, wherein the recessed feature comprises:
an engagement feature having a greater depth than one or more other engagement features of the pressure control tube; and/or
an uppermost engagement feature of the one or more engagement features of the pressure control tube.

10. The apparatus of claim 1, wherein the pressure control tube is in a fully lowered positioned when the releasable tube retaining means is in the substantially unstressed state.

11. The apparatus of claim 1, wherein the releasable tube retaining means comprises an elongate body having a coupled end and a free end, the coupled end configured to be coupled to a wall of a chamber of the pressure regulator device.

12. The apparatus of claim 11, wherein the coupled end is configured to be coupled to a top wall of the chamber and/or the coupled end comprises one or more coupling portions, preferably, the one or more coupling portions comprise hinge hooks or tabs that are configured to be pivotably coupled to the top wall.

13. The apparatus of claim 11, wherein the coupled end is configured to be pivotably coupled to the wall of the chamber.

14. The apparatus of claim 11, wherein the free end comprises an actuating member.

15. The apparatus of claim 14, wherein the actuating member is configured to be in an un-deformed position when the releasable tube retaining means is in the substantially unstressed state and/or the actuating member comprises a push tab.

16.-53. (canceled)

54. A releasable tube retaining means for retaining a pressure control tube in a user-defined position within a pressure regulator device, comprising:
a resiliently deformable locking member configured to be releasably engageable with an engagement feature of the pressure control tube, to retain the pressure control tube in a user-defined position;
wherein the releasable tube retaining means is configured to be adaptable between a first configuration in which the locking member is engaged with the engagement feature and resists movement of the pressure control tube, and a second configuration in which the locking member is at least partially disengaged from the engagement feature and the resistance to movement of the pressure control tube is reduced.

55. The releasable tube retaining means of claim 54, wherein when the releasable tube retaining means is in the second configuration, the resistance to movement of the pressure control tube is reduced in comparison to the resistance to movement of the pressure control tube when the releasable tube retaining means is in the first configuration.

56. The releasable tube retaining means of claim 54, wherein the locking member comprises a tube engagement portion configured to be releasably engageable with at least one of the engagement features.

57. The releasable tube retaining means of claim 56, wherein the tube engagement portion is configured to be at least partially disengaged from the engagement feature when the releasable tube retaining means is in the is in the second configuration.

58.-94. (canceled)

95. A pressure regulating device, comprising:
a chamber configured to hold a column of liquid,
a pressure control tube configured to be selectively moveable within the column of liquid to increase or decrease a pressure of the pressure regulating device, the pressure control tube comprising one or more engagement features spaced along a length of the pressure control tube, and
a releasable tube retaining means, comprising a first resiliently deformable locking member and a second resiliently deformable locking member, each of the first locking member and the second locking member comprising a tube engagement portion configured to be releasably engageable with at least one engagement feature;
wherein,
engagement of the tube engagement portion of the first locking member with an engagement feature of the one or more engagement features, and the tube engagement portion of the second locking member with an engagement feature of the one or more engagement features provides resistance to movement of the pressure control tube within the pressure regulating device and retains the pressure control tube at a user-defined depth within the column of liquid;
the releasable tube retaining means being selectively adaptable between:
a first configuration in which the tube engagement portions of the first locking member and the second locking member are each engaged with at least one of the one or more engagement features such that movement of the pressure control tube within the pressure regulating device is resisted by a first upstroke resistance force and a first downstroke resistance, and
a second configuration in which at least one of the tube engagement portions of the first locking member and the second locking member is at least partially disengaged from the engagement features, such that resistance to movement of the pressure control tube within the pressure regulator device is resisted by a second upstroke resistance force and a second downstroke resistance force which are less than the first upstroke resistance force and the first upstroke resistance force; and further wherein,
the releasable tube retaining means is adapted to be selectively engageable with the pressure control tube in a position in which the releasable tube retaining means is in a substantially unstressed state.
